(19) United States
(12) Patent Application Publication
(10) Pub. No.: US 2007/0259031 A1
(43) Pub. Date: Nov. 8, 2007

(54) COMPOSITIONS AND METHODS FOR CONVECTION ENHANCED DELIVERY OF HIGH MOLECULAR WEIGHT NEUROTHERAPEUTICS

(75) Inventors: Krystof S. Bankiewicz, Oakland, CA (US); Sandeep Kunwar, Hillsborough, CA (US)

Correspondence Address: JOHN P. O'BANION;O'BANION & RITCHEY LLP, 400 CAPITOL MALL SUITE 1550, SACRAMENTO, CA, 95814, US

(73) Assignee: THE REGENTS OF THE UNIVERSITY OF CALIFORNIA, Oakland, CA (US)

(21) Appl. No.: 11/740,508

(22) Filed: Apr. 26, 2007

Related U.S. Application Data
(60) Provisional application No. 60/795,371, filed on Apr. 26, 2006.
(60) Provisional application No. 60/900,492, filed on Feb. 9, 2007.

(51) Int. Cl. A61K 38/17; A61K 9/127

(52) U.S. Cl. 424/450; 514/2; 977/907

(57) ABSTRACT

A method of therapeutic treatment of CNS disorders using local convection enhanced delivery.

### CROSS-REFERENCE TO RELATED APPLICATIONS

This application claims priority to U.S. provisional patent application Ser. No. 60/795,371 filed on Apr. 26, 2006, incorporated herein by reference in its entirety, and to U.S. provisional patent application Ser. No. 60/900,492 filed on Feb. 9, 2007, incorporated herein by reference in its entirety.

### STATEMENT REGARDING FEDERALLY SPONSORED RESEARCH OR DEVELOPMENT

This invention was made with Government support under Grant No. P50 CA097257 awarded by the National Institutes of Health (NIH), and under Grant No. U54 NS045309 awarded by the National Institute of Neurological Disorders and Stroke (NINDS). The Government has certain rights in this invention.

### INCORPORATION-BY-REFERENCE OF MATERIAL SUBMITTED ON A COMPACT DISC

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. §1.14.

### BACKGROUND OF THE INVENTION

1. Field of the Invention

The present invention concerns disorders of the central nervous system. The invention relates specifically to the treatment of central nervous system disorders with high molecular weight neurotherapeutics delivered locally by convection enhanced delivery.

2. Description of Related Art

Disorders of the central nervous system (CNS) often result in serious morbidity, death or impairment of mobility because of the lack of effective surgical or medical therapies. Although potentially therapeutic compounds exist for treating many of these disorders, delivering effective doses of these agents selectively to target CNS tissue has remained a challenge. Systemic toxicity and an inability to cross the blood brain barrier frequently compromise the efficacy of compounds that exhibit promising activity in vitro. Additionally, many compounds that are capable of crossing the blood brain barrier exhibit non-uniform, inconsistent patterns of distribution as well as a frequent inability to effectively penetrate target tissue. Further, compounds delivered intraventricularly have also exhibited non-uniform distribution and poor target tissue penetration. Accordingly, the poor efficacy exhibited by therapeutic agents to date in respect of the treatment of CNS disorders may be due to administration and tissue distribution rather than the activity of agents per se.

Local delivery of therapeutics to the CNS is an alternative route of administration that overcomes many problems associated with systemic and intraventricular delivery in the treatment of CNS disorders. However, there are a number of limitations associated with direct infusion and diffusion-based delivery of therapeutics to target CNS regions, the most critical being a low tissue distribution volume. Clinical studies involving neurotrophin infusion into the brain parenchyma of patients with neurodegenerative disease have used continuous infusion and relied on diffusion for protein to reach target tissues. Without means for monitoring the distribution of infused neurotrophin, it is difficult to determine the therapeutic efficacy. For example, Gill et al. reported in Nat. Med. 9:5899-595, 2003 that, when glial cell line-derived neurotrophic factor (GDNF) was used in clinical studies as a treatment for Parkinson's disease, it was not clear how far GDNF would diffuse away from the catheter tip and that it is possible more rostral portions of the putamen would continue to degenerate if not reached by diffusion. They also found that, as the dose of GDNF was escalated, a high T2 MRI signal intensity was observed around the tip of the catheter, possibly owing to vasogenic edema or protein buildup, which required dosage reduction and potentially further compromised rostral portions of the putamen.

Convection-enhanced delivery (CED) is a promising technique for local delivery of therapeutics that overcomes some of the limitations inherent in diffusion-based delivery. Despite its promise, however, there are difficulties associated with CED. Many therapeutics do not appear amenable to convection-based delivery. For example, low molecular weight therapeutics are not readily convectible and show limited distribution with CED in CNS tissue, and failed attempts with large protein therapeutics have also been reported. Additionally, retrograde flow (reflux) along the catheter shaft and unwanted distribution of infusate to secondary sites is a reported problem. Reflux may cause infusate to reach unintended tissue and cause underexposure of the intended target. An additional factor influencing the distribution of a CED-delivered agent is the distribution of agent binding sites. It has been previously demonstrated that therapeutic growth factors delivered by CED exhibit limited distribution in the absence of a facilitating agent such as heparin. The facilitating agent appears to decrease binding of growth factor to binding sites in the infusate path, thereby increasing the tissue distribution volume of the growth factor. Finally, many therapeutic agents, particularly cytotoxic agents useful for the treatment of CNS tumors, are non-specific. The local delivery of such agents by CED or other methods, while providing an effective dose in target tissue and avoiding problems associated with systemic delivery, poses a threat to non-tumor CNS tissue exposed to infusate.

It will also be appreciated that therapeutics with reduced toxicity in respect of non-target tissue which retain therapeutic efficacy and the ability to be delivered locally by CED are highly desirable.

### SUMMARY OF INVENTION

The invention is directed to the therapeutic treatment of CNS disorders. The invention overcomes problems associated with many previous treatment regimens by employing local convection enhanced delivery. The invention additionally overcomes issues associated with local delivery, such as limited tissue distribution, unwanted binding site interaction and toxicity with the use of high molecular weight neurotherapeutics and, optionally, a facilitating agent. The invention stems in part from the finding that high molecular weight neurotherapeutics comprising active therapeutic agents may be convected in the CNS of large mammals and exhibit increased tissue distribution, decreased toxicity, and increased half-life as compared to corresponding low molecular weight active agents alone. Such high molecular weight neurotherapeutics may be used to achieve tissue concentrations of active agent up to many thousand fold higher than can be achieved with corresponding agent alone and with lower toxicity. The invention also derives from the important finding that high molecular weight neurotherapeutics may be convected in naturally occurring CNS tumor tissue of large mammals, a result that establishes the clinical applicability of CED as a means for administering high molecular weight neurotherapeutic in the treatment of CNS tumors.

In a preferred embodiment, the invention involves the use of a step-design reflux-free cannula, and thereby further addresses the issue of backflow associated with local delivery. In a highly preferred embodiment, the invention involves coadministration of a tracing agent, and thereby provides for real-time monitoring of high molecular weight neurotherapeutic distribution.

In accordance with the objectives outlined above, in one aspect, the invention provides high molecular weight neurotherapeutics locally deliverable by CED.

High molecular weight neurotherapeutics of the invention have a molecular weight greater than about 200 kDa, more preferably greater than about 500 kDa, more preferably greater than about 1000 kDa, more preferably greater than about 1500 kDa, more preferably greater than about 2000 kDa, more preferably greater than about 2500 kDa, more preferably greater than about 3000 kDa, more preferably greater than about 3500 kDa, more preferably greater than about 4000 kDa, more preferably greater than about 4500 kDa, more preferably greater than about 5000 kDa, more preferably greater than about 5500 kDa, more preferably greater than about 6000 kDa, more preferably greater than about 6500 kDa, more preferably greater than about 7000 kDa, more preferably greater than about 7500 kDa, more preferably greater than about 8000 kDa, more preferably greater than about 8500 kDa, more preferably greater than about 9000 kDa, more preferably greater than about 9500 kDa, and more preferably greater than about 10000 kDa.

In one embodiment, a high molecular weight neurotherapeutic of the invention has a diameter or length greater than about 10 nm, more preferably greater than about 25 nm, more preferably greater than about 40 nm, more preferably greater than about 50 nm, more preferably greater than about 60 nm, more preferably greater than about 70 nm, more preferably greater than about 80 nm, more preferably greater than about 90 nm, more preferably greater than about 100 nm, more preferably greater than about 110 nm, and more preferably greater than about 120 nm. In some embodiments, a high molecular weight neurotherapeutic of the invention has a diameter or length greater than about 130 nm, or greater than about 140 nm, or greater than about 150 nm, or greater than about 160 nm, or greater than about 170 nm, or greater than about 180 nm, or greater than about 190 nm, or greater than about 200 nm.

High molecular weight neurotherapeutic compositions of the invention comprise an active agent and a carrier.

In one embodiment, the carrier is a synthetic carrier.

A wide variety of synthetic carriers are available for use in the high molecular weight neurotherapeutics of the invention. In a preferred embodiment, the carrier is a liposome. In another preferred embodiment, the carrier is a metal particle, such as a gold particle, or a polymer.

In one embodiment, the carrier is a naturally occurring composition or variant thereof. Examples of such carriers include virus particles, including modified virus particles (e.g., those having a modified surface protein profile).

In one embodiment, the high molecular weight neurotherapeutic is larger than an AAV virus.

In one embodiment, the high molecular weight neurotherapeutic has a higher molecular weight than an AAV virus.

In one embodiment, the high molecular weight neurotherapeutic comprises a carrier other than AAV.

A wide variety of active agents are available for use in the high molecular weight neurotherapeutics of the invention. The active agent will be capable of effecting a desirable response in target tissue. For example, active agents capable of effecting a desirable response in target tissue that comprises tumor cells include cytotoxic agents. The nature of the invention is such that the nature of the active agent is not limited by the means of delivery.

In a preferred embodiment, the active agent is a nucleic acid, a protein, or a small molecule chemical compound.

In one embodiment, the active agent is a small molecule chemical compound capable of modulating the activity of an enzyme.

In one embodiment, the active agent is a small molecule chemical compound capable of modulating the activity of a protein kinase or phosphatase.

In one embodiment, the active agent is a small molecule chemical compound capable of modulating the activity of a lipid kinase or phosphatase.

In one embodiment, the active agent is a therapeutic nucleic acid.

In a preferred embodiment, the therapeutic nucleic acid is an antisense nucleic acid, an siRNA, a short hairpin RNA, or an enzymatic nucleic acid.

In one embodiment, the active agent is an antibody.

In one embodiment, the high molecular weight neurotherapeutic when administered has a V_{d}:Vᵢ ratio of 1:1 or greater.

In one aspect, the invention provides pharmaceutical compositions comprising high molecular weight neurotherapeutics disclosed herein.

In one embodiment, a pharmaceutical composition comprises a high molecular weight neurotherapeutic, wherein the pharmaceutical composition is deliverable by CED to the CNS of a patient having a CNS disorder, wherein the high molecular weight neurotherapeutic is present in an amount sufficient to provide a therapeutically effective dose when the pharmaceutical composition is delivered by CED to the CNS of the patient.

In a preferred embodiment, a pharmaceutical composition further comprises a tracing agent.

In a preferred embodiment, the tracing agent is an MRI contrast agent, sometimes referred to herein as an “MRI magnet”.

In a preferred embodiment, the tracing agent comprises a liposome. In an especially preferred embodiment, the tracing agent comprises a liposome containing an MRI magnet, preferably gadolinium chelate. In a highly preferred embodiment, the tracing agent consists essentially of a liposome containing an MRI magnet, preferably gadolinium chelate.

In one embodiment, a pharmaceutical composition comprises a facilitating agent in addition to a high molecular weight neurotherapeutic.

In one embodiment, in addition to a high molecular weight neurotherapeutic, a pharmaceutical composition comprises means for modifying osmotic pressure in vivo and facilitating movement of the high molecular weight neurotherapeutic. Preferred means include mannitol.

In one aspect, the invention provides kits for the treatment of CNS disorders, which kits comprise one or more pharmaceutical compositions of the invention. In one embodiment, a kit of the invention further comprises a delivery device useful for CED, preferably a cannula, and more preferably a step-design reflux-free cannula. In one embodiment, a kit of the invention further comprises a pump useful for CED.

In one aspect, the invention provides methods for CED of high molecular weight neurotherapeutics to target CNS tissues. CED is preferably done in conjunction with a step-design reflux-free cannula. In its most preferred embodiment, the method further involves coadministration of a tracing agent which provides for guided delivery.

In one embodiment, the method involves the use of a facilitating agent.

In one aspect, the invention provides methods for delivering a high molecular weight neurotherapeutic to target CNS tissue in a subject, comprising CED of a high molecular weight neurotherapeutic via the perivascular space. In one embodiment, the target CNS tissue is remote to the CNS infusion site.

In one embodiment, the method involves coadministration of a composition having cerebral vasomotor properties in order to optimize the delivery and distribution of a high molecular weight neurotherapeutic.

In one embodiment, the invention provides methods for delivering a high molecular weight neurotherapeutic comprising a viral-based carrier to a location which is not achievable by axonal transport of the high molecular weight neurotherapeutic from the infusion site. In one embodiment, the target CNS tissue is remote to the CNS infusion site.

In one aspect, the invention provides methods for treating a subject having a CNS tumor. The methods comprise delivering a therapeutically effective amount of a pharmaceutical composition of the invention to a subject having a CNS tumor.

In a preferred embodiment, CED is done in conjunction with a step-design reflux-free cannula. In its most preferred embodiment, the method further involves coadministration of a tracing agent which provides for guided delivery.

In one aspect, the invention provides methods for reducing the growth of a tumor cell in the CNS of a subject. The methods comprise delivering a high molecular weight neurotherapeutic of the invention to a tumor cell in the CNS of a subject by CED, wherein the high molecular weight neurotherapeutic reduces the growth of the tumor cell.

In one embodiment, the tumor cell is internal to the outer margin of a tumor in which it is located.

In one aspect, the invention provides methods for reducing the survival of a tumor cell in the CNS of a subject. The methods comprise delivering a high molecular weight neurotherapeutic of the invention to a tumor cell in the CNS of a subject by CED, wherein the high molecular weight neurotherapeutic reduces the survival of the tumor cell.

In one embodiment, the tumor cell is internal to the outer margin of a tumor in which it is located.

In one aspect, the invention provides methods for inhibiting cell cycle progression of a tumor cell in the CNS of a subject. The methods comprise delivering a high molecular weight neurotherapeutic of the invention to a tumor cell in the CNS of a subject by CED, wherein the high molecular weight neurotherapeutic reduces cell cycle progression in the tumor cell.

In one embodiment, the tumor cell is internal to the outer margin of a tumor in which it is located.

In one aspect, the invention provides methods for promoting the survival of a neuron responsive to an active agent, comprising delivering a high molecular weight neurotherapeutic comprising such an active agent to such a responsive neuron in the CNS of a subject by CED, wherein the high molecular weight neurotherapeutic promotes survival of the neuron.

In a preferred embodiment, the subject has a CNS disorder, which disorder is associated with neuronal death and/or dysfunction at a locus comprising the responsive neuron. In a preferred embodiment, the disorder is a neurodegenerative disease. In another embodiment, the disorder is stroke. In another embodiment, the disorder is cancer.

In one aspect, the invention provides methods for promoting a particular phenotype of a neuron responsive to an active agent, comprising delivering a high molecular weight neurotherapeutic comprising such an active agent to such a responsive neuron in the CNS of a subject by CED, wherein the high molecular weight neurotherapeutic promotes or maintains the phenotype in the neuron.

In one aspect, the invention provides methods for modulating synapse formation of a neuron responsive to an active agent, comprising delivering a high molecular weight neurotherapeutic comprising such an active agent to such a responsive neuron in the CNS of a subject by CED, wherein the high molecular weight neurotherapeutic modulates synapse formation in the neuron.

In one aspect, the invention provides methods for modulating electrical activity of a neuron responsive to an active agent, comprising delivering a high molecular weight neurotherapeutic comprising such an active agent to such a responsive neuron in the CNS of a subject by CED, wherein the high molecular weight neurotherapeutic modulates the electrical activity in the neuron.

In one embodiment, an active agent acts on a responsive neuron secondarily by first eliciting a response from another cell in the CNS. In a preferred embodiment, an active agent acts directly on a responsive neuron.

In the methods of the invention, a high molecular weight neurotherapeutic is preferably delivered in the form of a pharmaceutical composition disclosed herein.

Many methods of the invention comprise CED. In a preferred embodiment, CED comprises an infusion rate of between about 0.5 μL/min and about 10 μL/min.

In a preferred embodiment, CED comprises an infusion rate of greater than about 0.5 μL/min, more preferably greater than about 0.7 μL/min, more preferably greater than about 1 μL/min, more preferably greater than about 1.2 μL/min, more preferably greater than about 1.5 μL/min, more preferably greater than about 1.7 μL/min, more preferably greater than about 2 μL/min, more preferably greater than about 2.2 μL/min, more preferably greater than about 2.5 μL/min, more preferably greater than about 2.7 μL/min, and more preferably greater than about 3 μL/min, as well as preferably less than about 25 μL/min, more preferably less than 20 μL/min, more preferably less than about 15 μL/min, more preferably less than about 12 μL/min, and more preferably less than about 10 μL/min.

In a preferred embodiment, CED comprises incremental increases in flow rate, referred to as “stepping”, during delivery. Preferably, stepping comprises infusion rates of between about 0.5 μL/min and about 10 μL/min.

In a preferred embodiment, stepping comprises infusion rates of greater than about 0.5 μL/min, more preferably greater than about 0.7 μL/min, more preferably greater than about 1 μL/min, more preferably greater than about 1.2 μL/min, more preferably greater than about 1.5 μL/min, more preferably greater than about 1.7 μL/min, more preferably greater than about 2 μL/min, more preferably greater than about 2.2 μL/min, more preferably greater than about 2.5 μL/min, more preferably greater than about 2.7 μL/min, and more preferably greater than about 3 μL/min, as well as preferably less than about 25 μL/min, more preferably less than 20 μL/min, more preferably less than about 15 μL/min, more preferably less than about 12 μL/min, and more preferably less than about 10 μL/min.

In a preferred embodiment, CED is performed with the use of a CED-compatible reflux-free step-design cannula. An especially preferred cannula is disclosed in Krauze et al., J Neurosurg. November 2005 ;103(5):923-9, incorporated herein by reference in its entirety, as well as in U.S. Patent Application Publication No. US 2007/0088295 A1, incorporated herein by reference in its entirety, and United States Patent Application Publication No. US 2006/0135945 A1, incorporated herein by reference in its entirety.

In one embodiment, the step-design cannula is compatible with chronic administration. In another embodiment, the step-design cannula is compatible with acute administration.

Treatment methods herein preferably involve preoperative diagnosis.

In one embodiment, preoperative diagnosis involves genetic screening.

In a preferred embodiment, preoperative diagnosis involves neuroimaging. Preferably, the neuroimaging done involves PET, SPECT, MRI, X-ray computed tomography, or a combination thereof.

Treatment methods herein also preferably comprise neuroimaging, preferably MRI, for target localization and guided cannula placement. Preferably a stereotactic holder is used in conjunction with neuroimaging to provide for guided cannula placement at or proximal to a target neuronal population.

Treatment methods herein also preferably comprise neuroimaging for monitoring infusate distribution. In a preferred embodiment, a treatment method comprises the use of MRI in conjunction with an administered MRI magnet for monitoring infusate distribution.

Methods of producing a pharmaceutical composition of the invention are also provided.

In one aspect, the invention provides a delivery device comprising a pharmaceutical composition of the invention.

In one aspect, the invention provides a catheter or cannula comprising a pharmaceutical composition of the invention.

In one aspect, the invention provides a delivery device comprising a pump that is capable of effecting delivery of a pharmaceutical composition of the invention by CED. In a preferred embodiment, the device further comprises a pharmaceutical composition of the invention. In a preferred embodiment, the device further comprises a CED-compatible, reflux-free step-design cannula, which cannula is compatible with chronic or acute administration.

Another aspect of the invention involves methods of producing a medicament useful for the treatment of a CNS disorder. In one embodiment, the medicament is a high molecular weight therapeutic. In one embodiment, the medicament is deliverable by CED to the CNS of a patient. In one embodiment, the high molecular weight therapeutic comprises a carrier and an active agent. In one embodiment, the carrier is a synthetic carrier. In one embodiment, the synthetic carrier is a liposome. In one embodiment, the high molecular weight neurotherapeutic has a molecular weight greater than about 200 kDa. In one embodiment, the high molecular weight neurotherapeutic has a diameter or length greater than about 10 nm. in one embodiment, the high molecular weight neurotherapeutic comprises an active agent selected from the group consisting of nucleic acids, proteins, and small molecule chemical compounds. In one embodiment, the CED to the CNS is performed with a V_{d}:Vᵢ greater than 1:1. In one embodiment, the medicament further comprises a tracing agent. in one embodiment, the tracing agent is an MRI magnet. In one embodiment, the MRI magnet is gadolinium chelate. In one embodiment, the CNS disorder is an acute CNS disorder. In one embodiment, the CNS disorder is a chronic CNS disorder. In one embodiment, the CNS disorder is cancer. In one embodiment, the CNS disorder is a neurodegenerative disease. In one embodiment, the active agent is selected from the group consisting of antineoplastic agents, radioiodinated compounds, toxins (including protein toxins), cytostatic or cytolytic drugs, genetic and viral vectors, vaccines, synthetic vectors, growth factors, neurotrophic factors, hormones, cytokines, enzymes and agents for targeted lesioning of specific sites. In one embodiment, the active agent is selected from the group consisting of nucleic acids, nucleic acid analogs, proteins, including antibodies, small molecule chemical compositions, agents that exhibit toxicity and unwanted effects when administered systemically, EGFR inhibitors, Tarceva, Iressa, topoisomerase inhibitors, irinotecan (CPT-11), etoposide, topotecan, edotecarin, rubitican, valrubicin, fostriecin, GL331, XR5000, SGN15, anthrcyclines, doxorubicin, alkylating agents, temaxolamide, carboplatin, cisplatin, dacarbazine (DTIC), mTOR inhibitors, Rapamycin, CCI-779, RAD 001, Farnasyl transferase inhibitors, R11577, lonafarnib; growth factor inhibitors, tyrosine kinase inhibitors, AEE788, SU5416, erlotinab, ZD1839, Enzastaurin, lapatinib, AP23573, sorafenib, ST1571 (Gleevac), PTK787, vatalanib, semaxanib, PKI166, quercetin, BIBX1382, Mubritinib, Erbstatin, RG13022, RG13291, AG1295, leflunomide, Gefitinib, HDAC inhibitors, depsipetide, integrin inhibitors, celengitide, COX-2 inhibitors, everolimus, vioxx, celebrex, telomerase inhibitors, grn 163, TGFb inhibitors, MDMA inhibitors, AMPA inhibitors, GABA, GABA agonists, inhibitors of axonal sprouting, and combinations thereof, including combinations of mTOR inhibitor and tyrosine kinase inhibitor, which combinations may be in a single carrier.

Further aspects of the invention will be brought out in the following portions of the specification, wherein the detailed description is for the purpose of fully disclosing preferred embodiments of the invention without placing limitations thereon.

### BRIEF DESCRIPTION OF THE DRAWINGS

The invention will be more fully understood by reference to the following drawings which are for illustrative purposes only:

FIG. 1 is a series of images showing convective delivery of CPT-11 liposomes and gadolinium chelate liposomes (tracer liposomes) in a dog with spontaneous grade IIII astrocytoma.

FIG. 2 illustrates tumor mass penetration by CPT-11 liposomes and gadolinium chelate liposomes.

FIG. 3 illustrates tumor mass penetration by CPT-11 liposomes and gadolinium chelate liposomes. Tumor on left, liposomes on right.

FIG. 4 illustrates tumor mass penetration by CPT-11 liposomes and gadolinium chelate liposomes. Tumor on bottom left panel.

FIG. 5 is a graph of Vd vs. Vi for tumor infusion.

FIG. 6 illustrates distribution Corona Radiata Dog vs. Tumor Dog

FIG. 7 shows imaging of convective delivery of gadolinium chelate liposomes and gadolinium chelate liposomes plus liposomal topotecan into canine tumor tissue (right, liposomal gadolinium (Gd); left, Gd+liposomal topotecan (LS topo)).

FIG. 8 shows imaging of convective delivery of gadolinium chelate liposomes and gadolinium chelate liposomes plus liposomal topotecan into canine tumor tissue (right, Gd only; left, Gd+LS topo).

FIG. 9 shows imaging of convective delivery of gadolinium chelate liposomes and gadolinium chelate liposomes plus liposomal topotecan into canine tumor tissue (right, Gd only; left, Gd+LS topo).

FIG. 10 shows imaging of convective delivery of gadolinium chelate liposomes and gadolinium chelate liposomes plus liposomal topotecan into canine tumor tissue (right, Gd only; left, Gd+LS topo).

FIG. 11 is a graph of Vd vs. Vi for tumor infusion.

FIG. 12 is a graph of Vd vs. Vi for astrocytoma grade III and oligodendroglioma tumor infusion.

FIG. 13 illustrates infusion of a mixture of liposomal CPT-11 and GDL into a temporal lobe astrocytoma in a canine patient (case #1).

FIG. 14 illustrates tumor growth arrest following infusion of a mixture of liposomal CPT-11 and GDL into a temporal lobe astrocytoma in a canine patient (case #1).

FIG. 15 shows the relationship between volume of infusion and volume of distribution in canine brain tumors.

FIG. 16 shows MR imaging and neuropathological results of CED of CPT-11/liposome/gadolinium delivered intratumorally into the canine diffuse astrocytoma in the right piriform lobe. A. MR image of astrocytoma before, B. immediately post infusion and C. 3 months later with marked reduction in tumor size from three infusions. D. Brain section with necrosis, and residual tumor E. and F. illustrating the area of necrosis (N), tumor infusion (I) and intact non-infused tumor (T) (case #1).

FIG. 17 illustrates infusion of a mixture of liposomal CPT-11 and GDL into a frontal/parietal lobe anaplastic oligodendroglioma grade III tumor in a canine patient (case #2).

FIG. 18 shows MR imaging of tumor dog diagnosed with pyriform lobe grade III astrocytoma. Panels A,B,C represent conclusion of simultaneous infusion into 3 sites. Majority of the tumor was covered by the CPT11/GDL. Consistent ratio of Vi/Vd)that was seen in other 2 cases is demonstrated. Tumor volume was reduced 3 months after infusion shown in Panels A, B, C. (case #3).

### DETAILED DESCRIPTION OF INVENTION

The invention provides compositions and methods for delivering high molecular weight neurotherapeutics to target tissues of the CNS by convection enhanced delivery (CED). In a preferred embodiment, the invention provides compositions and methods for guided delivery of high molecular weight neurotherapeutics, which involve the use of a tracing agent. The use of a tracing agent provides for real-time monitoring of the distribution and concentration of a high molecular weight neurotherapeutic, thereby increasing the safety and efficacy with which active agents may be delivered to CNS tissues.

By CNS disorder is meant a disorder of the central nervous system of a subject. The disorder may be associated with the death and/or dysfunction of a particular neuronal population in the CNS. The disorder may be an acute or chronic disorder of the CNS. The disorder may be associated with the aberrant growth of cells within the CNS. The aberrantly growing cells of the CNS may be native to the CNS or derived from other tissues. Included among CNS disorders are cancer, infection, head trauma, spinal cord injury, multiple sclerosis, dementia with Lewy bodies, ALS, lysosomal storage disorders, psychiatric disorders, neurodegenerative diseases, stroke, epilepsy, psychiatric disorders, disorders of hormonal balance. Further contemplated are methods for reducing inflammation that is associated with a CNS disorder characterized by neuronal death and/or dysfunction.

The term “subject” refers to large mammals, preferably primates, and most preferably humans, and does not include small mammals such as rodents. A “subject” of the invention is a mammal capable of receiving an infusate composition of the invention.

A high molecular weight neurotherapeutic of the invention comprise an active agent and a carrier. In one embodiment, the carrier is a synthetic carrier. In another embodiment, the carrier is a naturally occurring composition or variant thereof.

In a preferred embodiment, a high molecular weight neurotherapeutic of the invention consists essentially of an active agent and a carrier.

High molecular weight neurotherapeutics of the invention have a molecular weight greater than about 200 kDa, more preferably greater than about 500 kDa, more preferably greater than about 1000 kDa, more preferably greater than about 1500 kDa, more preferably greater than about 2000 kDa, more preferably greater than about 2500 kDa, more preferably greater than about 3000 kDa, more preferably greater than about 3500 kDa, more preferably greater than about 4000 kDa, more preferably greater than about 4500 kDa, more preferably greater than about 5000 kDa, more preferably greater than about 5500 kDa, more preferably greater than about 6000 kDa, more preferably greater than about 6500 kDa, more preferably greater than about 7000 kDa, more preferably greater than about 7500 kDa, more preferably greater than about 8000 kDa, more preferably greater than about 8500 kDa, more preferably greater than about 9000 kDa, and more preferably greater than about 9500 kDa, more preferably greater than about 10000 kDa.

In one embodiment, a high molecular weight neurotherapeutic of the invention has a diameter or length greater than about 10 nm, more preferably greater than about 25 nm, more preferably greater than about 40 nm, more preferably greater than about 50 nm, more preferably greater than about 60 nm, more preferably greater than about 70 nm, or greater than about 80 nm, more preferably greater than about 90 nm, more preferably greater than about 100 nm, more preferably greater than about 110 nm, and more preferably greater than about 120 nm. In some embodiments, a high molecular weight neurotherapeutic of the invention has a diameter or length greater than about 130 nm, or greater than about 140 nm, or greater than about 150 nm, or greater than about 160 nm, or greater than about 170 nm, or greater than about 180 nm, or greater than about 190 nm, or greater than about 200 nm.

A carrier is a composition that may be used in combination with an active agent, and optional other components, to produce a high molecular weight neurotherapeutic which is locally deliverable by CED.

A high molecular weight neurotherapeutic locally deliverable by CED is a neurotherapeutic that is capable of being delivered locally by CED in the CNS of a subject, preferably a canine or a primate, and most preferably a human.

As used herein, “active agent” or “therapeutic agent” refers to any molecule that may be delivered to CNS target tissue in the form of a high molecular weight neurotherapeutic, and when so delivered, effects a desirable response in the target CNS tissue. Therapeutic agents include antineoplastic agents, radioiodinated compounds, toxins (including protein toxins), cytostatic or cytolytic drugs, genetic and viral vectors, vaccines, synthetic vectors, growth factors, neurotrophic factors, hormones, cytokines, enzymes and agents for targeted lesioning of specific sites. Therapeutic agents include, but are not limited to, nucleic acids, including nucleic acid analogs, proteins, including antibodies, and small molecule chemical compositions. Active agents include agents that exhibit toxicity and unwanted effects when administered systemically. Especially preferred active agents include EGFR inhibitors, including Tarceva, Iressa; topoisomerase inhibitors, preferably selected from irinotecan (CPT-11), etoposide, topotecan, edotecarin, rubitican, valrubicin, fostriecin, GL331, XR5000, SGN15; anthrcyclines, including doxorubicin; alkylating agents, including temaxolamide, carboplatin, cisplatin, dacarbazine (DTIC); mTOR inhibitors, including Rapamycin, CCI-779, RAD 001; Farnasyl transferase inhibitors, including R11577, lonafarnib; growth factor inhibitors, including tyrosine kinase inhibitors, including AEE788, SU5416, erlotinab, ZD1839, Enzastaurin, lapatinib, AP23573, sorafenib, ST1571 (Gleevec), PTK787, vatalanib, semaxanib, PKI166, quercetin, BIBX1382, Mubritinib, Erbstatin, RG13022, RG13291, AG1295, leflunomide, Gefitinib; HDAC inhibitors, including depsipetide; integrin inhibitors, including celengitide; COX-2 inhibitors, including everolimus, vioxx, celebrex; telomerase inhibitors, including grn 163; TGFβ inhibitors; MDMA inhibitors; AMPA inhibitors; GABA; GABA agonists; inhibitors of axonal sprouting; and combinations thereof, including combinations of mTOR inhibitor and tyrosine kinase inhibitor, which combinations may be in a single carrier.

For further discussion of the use of growth factors in high molecular weight neurotherapeutics deliverable by CED, see U.S. provisional application Ser. No. 60/795,012, filed on Apr. 25, 2006, which is expressly incorporated herein in its entirety by reference.

A therapeutic infusate composition is a volume of pharmaceutical composition to be delivered by CED in a single administration. The volume of infusate will be largely determined by the target tissue and its volume. Typical volumes will be between about 10 μl and about 10 cc, though larger (particularly for brain tumors) and smaller volumes may be used.

The term “target tissue” refers to a physical (usually anatomical) target in the CNS. Examples of target tissues include a tumor, such as a brain tumor, a cyst, a seizure focus in the brain to be ablated, or a particular neuroanatomic substructure (such as the pons, midbrain, basal forebrain, striatum, thalamus, optic tract or occipital cortex). The target tissue may be an entire physical target or some portion thereof to which delivery of a therapeutic agent is desired.

A tracing agent is preferably detectable by magnetic resonance imaging (MRI) or X-ray computed tomography. The distribution of tracing agent is monitored and used as an indirect measure of the distribution of high molecular weight neurotherapeutic. This monitoring is done to verify that the high molecular weight neurotherapeutic is reaching target tissue and achieving an effective concentration therein and to detect unwanted delivery of infusate to non-target tissue.

In a preferred embodiment, a tracing agent is separate from the high molecular weight neurotherapeutic. The tracing agent is distributed in a manner that correlates with that of the high molecular weight neurotherapeutic and thus is an indirect indicator of high molecular weight neurotherapeutic distribution.

In a preferred embodiment, the tracing agent and the high molecular weight neurotherapeutic each comprise the same type of carrier, which confers highly similar distribution characteristics thereto.

In a highly preferred embodiment, the tracing agent and the high molecular weight neurotherapeutic comprise liposomes. Liposome-based tracing agents are very highly accurate indirect indicators of the distribution of liposome-based high molecular weight neurotherapeutics. Further, the use of liposomes (i) reduces the interaction of an active agent with binding sites in CNS tissue and thereby increases its distribution; (ii) reduces toxicity of many active agents, allowing for a much higher tissue concentration of active agent; and (iii) increases tissue residency time of an active agent.

The act of “monitoring” refers to obtaining serial images of the tracing agent over time. By monitoring the distribution of the tracing agent, the location and volume of distribution of the high molecular weight neurotherapeutic within the tissue may be determined at any time during the infusion process. Serial images may be obtained at any rate up to the maximum rate that the imaging instrument can obtain images. For example, serial images may be obtained at intervals ranging from a few milliseconds to hours, but more typically at intervals of minutes, such as intervals of 1, 2, 5, 10, 15, 20 or 30 minutes. The interval between serial images may be varied during infusion. In some instances, it may be desirable to obtain images at short intervals (for example, every 5, 10, or 15 seconds) at the beginning of the infusion process to detect backflow along the cannula, or to verify that the infusate is entering the desired target tissue. Once delivery to the proper site is confirmed, the interval between images may be lengthened, and the images used to follow the progress of infusion.

In one aspect, the invention provides treatment methods that comprise delivering a pharmaceutical composition of the invention by CED, wherein the pharmaceutical composition comprises a tracing agent, monitoring the distribution of the tracing agent as it moves through the CNS, and ceasing delivery of the pharmaceutical composition when the high molecular weight neurotherapeutic is distributed in a predetermined volume within the CNS. The movement of the tracing agent through the solid tissue may be monitored by an imaging technique such as magnetic resonance imaging (MRI) or X-ray computed tomography (CT). The tracing agent has a mobility in CNS tissue that is substantially similar to the therapeutic agent, and delivery is ceased when the tracing agent is observed to reach a desired region or achieve a desired volume of distribution, or to reach or nearly reach or exceed the borders of the target tissue.

The predetermined volume may correspond with the volume occupied by a tumor, or the predetermined volume may be a particular region of the brain that is targeted for destruction (e.g. the medial globus pallidus). In one embodiment the predetermined volume exceeds the volume of a CNS tumor. In another embodiment, the predetermined volume is less than the volume of a CNS tumor. The predetermined volume of distribution is “substantially similar” to the volume of distribution observed for a tracing agent that is being monitored to follow the infusion. “Substantially similar” refers to a difference in volume of less than 20%. More preferably, the difference in volume is less than 15%, more preferably less than 10%, more preferably less than 5%. By monitoring the distribution of the tracing agent, infusion may be ceased when the predetermined volume of distribution is reached.

Volume of distribution may be determined, for example, by using imaging software that is standard in the art, e.g., iFLOW™. See also, for example, Krautze et al., Brain Res. Protocols, 16:20-26, 2005; and Saito et al., Exp. Neurol., 196:3891-389, 2005, each of which is incorporated herein by reference in its entirety.

A tracer preferably comprises a paramagnetic ion for use with MRI. Suitable metal ions include those having atomic numbers of 22-29 (inclusive), 42, 44 and 58-70 (inclusive) and have oxidation states of +2 or +3. Examples of such metal ions are chromium (III), manganese (II), iron (II), iron (III), cobalt (II), nickel (II), copper (II), praseodymium (III), neodymium (III), samarium (III), gadolinium (III), terbium (III), dysprosium (III), holmium (III), erbium (III) and ytterbium (III).

In embodiments wherein X-ray imaging (such as CT) is used to monitor CED, the tracer may comprise a radiopaque material. Suitable radiopaque materials are well known and include iodine compounds, barium compounds, gallium compounds, thallium compounds, and the like. Specific examples of radiopaque materials include barium, diatrizoate, ethiodized oil, gallium citrate, iocarmic acid, iocetamic acid, iodamide, iodipamide, iodoxamic acid, iogulamide, iohexol, iopamidol, iopanoic acid, ioprocemic acid, iosefamic acid, ioseric acid, iosulamide meglumine, iosumetic acid, iotasul, iotetric acid, iothalamic acid, iotroxic acid, ioxaglic acid, ioxotriroic acid, ipodate, meglumine, metrizamide, metrizoate, propyliodone, and thallous chloride.

High Molecular Weight Neurotherapeutics

High molecular weight neurotherapeutics of the invention have a molecular weight greater than about 200 kDa, more preferably greater than about 500 kDa, more preferably greater than about 1000 kDa, more preferably greater than about 1500 kDa, more preferably greater than about 200 kDa, more preferably greater than about 2500 kDa, more preferably greater than about 3000 kDa, more preferably greater than about 3500 kDa, more preferably greater than about 4000 kDa, more preferably greater than about 4500 kDa, more preferably greater than about 5000 kDa, more preferably greater than about 5500 kDa, more preferably greater than about 6000 kDa, more preferably greater than about 6500 kDa, more preferably greater than about 7000 kDa, more preferably greater than about 7500 kDa, more preferably greater than about 8000 kDa, more preferably greater than about 8500 kDa, more preferably greater than about 9000 kDa, more preferably greater than about 9500 kDa, and more preferably greater than about 10000 kDa.

In one embodiment, a high molecular weight neurotherapeutic of the invention has a diameter or length greater than about 10 nm, more preferably greater than about 20 nm, more preferably greater than about 30 nm, more preferably greater than about 40 nm, more preferably greater than about 50 nm, more preferably greater than about 60 nm, more preferably greater than about 70 nm, more preferably greater than about 80 nm, more preferably greater than about 90 nm, more preferably greater than about 100 nm, more preferably greater than about 110 nm, and more preferably greater than about 120 nm. In some embodiments, a high molecular weight neurotherapeutic of the invention has a diameter or length greater than about 130 nm, or greater than about 140 nm, or greater than about 150 nm, or greater than about 160 nm, or greater than about 170 nm, or greater than about 180 nm, or greater than about 190 nm, or greater than about 200 nm.

High molecular weight neurotherapeutic compositions of the invention comprise an active agent and a carrier.

In one embodiment, the carrier is a synthetic carrier.

A wide variety of synthetic carriers are available for use in the high molecular weight neurotherapeutics of the invention. In a preferred embodiment, the carrier is a liposome. In another preferred embodiment, the carrier is a metal particle, such as a gold particle, or a polymer. Regarding carriers, see, for example, Felgner et al., Ann N Y Acad Sci. Nov. 27, 1995;772:126-39; Ramsay et al., Curr Drug Deliv. October 2005;2(4):341-51; Allen et al., Anticancer Agents Med Chem. November 2006;6(6):513-23; Mitra et al., Curr Pharm Des. 2006;12(36):4729-49, each of which is incorporated herein by reference in its entirety.

In one embodiment, the carrier is a naturally occurring composition or variant thereof. Examples of such carriers include virus particles, including modified virus particles (e.g., those having a modified surface protein profile). For example, see de Jonge et al., Gene Therapy (2006) 13, 400-411, incorporated herein by reference in its entirety.

In one embodiment, the high molecular weight neurotherapeutic is larger than an AAV virus.

In one embodiment, the high molecular weight neurotherapeutic has a higher molecular weight than an AAV virus.

In one embodiment, the high molecular weight neurotherapeutic comprises a carrier other than AAV.

Administration

In the methods herein, pharmaceutical compositions comprising high molecular weight neurotherapeutics are locally delivered to a target CNS population by convection enhanced delivery (“CED”). By “CED” is meant infusion at a rate greater than 0.5 μl/min. In a preferred embodiment, high molecular weight neurotherapeutic is delivered by CED through a suitable catheter or cannula, preferably a step-design reflux-free cannula. The method involves positioning the tip of the cannula at least in close proximity to the target tissue. After the cannula is positioned, it is connected to a pump which delivers the neurotherapeutic through the cannula tip to the target tissue. A pressure gradient from the tip of the cannula is maintained during infusion.

By “proximal to” a target population is meant within an effective distance of the target population. In particular, with respect to the positioning of a cannula relative to target tissue, proximity refers to a distance such that infusate will reach the target tissue when delivered by CED.

In a preferred embodiment, a step-design reflux-free cannula is joined with a pump that produces enough pressure to cause the high molecular weight neurotherapeutic to flow through the cannula to the target tissue at controlled rates. Any suitable flow rate can be used such that the intracranial pressure is maintained at suitable levels so as not to injure the brain tissue. More than a single cannula can be used. Penetration of the high molecular weight neurotherapeutic into target tissue is greatly facilitated by positive pressure infusion over a period of hours.

In one embodiment, penetration is further augmented by the use of a facilitating agent, such as low molecular weight heparin.

In a highly preferred embodiment, a tracing agent, preferably an MRI magnet, is co-delivered with the high molecular weight neurotherapeutic to provide for real-time monitoring of tissue distribution of infusate. Use of a tracing agent may inform the cessation of delivery.

Any suitable amount of high molecular weight neurotherapeutic can be administered in this manner. Suitable amounts are amounts that are therapeutically effective without causing an overabundance of undesirable side effects. In practice, the amount of high molecular weight neurotherapeutic will depend on the nature of the target tissue (e.g., necrosis associated with tumors or stroke; trophically deprived cells and damaged tissue, as in neurodegenerative disease), the nature of the active agent (e.g., antitumor agent, or growth factor), the volume of the target tissue, and additional factors, as recognized by one of skill in the art. The Vᵢ:V_{d} ratio of high molecular weight neurotherapeutic when administered to the CNS by CED Is great than or equal to 1:1. The ratio varies to between regions of the CNS, and Vᵢ will be adjusted accordingly without undue experimentation.

In a preferred embodiment, CED comprises an infusion rate of between about 0.5 μL/min and about 10 μL/min.

Though less preferred, rates less than 0.5 μl may be used.

In a preferred embodiment, CED comprises an infusion rate of greater than about 0.5 μL/min, more preferably greater than about 0.7 μL/min, more preferably greater than about 1 μL/min, more preferably greater than about 1.2 μL/min, more preferably greater than about 1.5 μL/min, more preferably greater than about 1.7 μL/min, more preferably greater than about 2 μL/min, more preferably greater than about 2.2 μL/min, more preferably greater than about 2.5 μL/min, more preferably greater than about 2.7 μL/min, more preferably greater than about 3 μL/min, as well as preferably less than about 25 μL/min, more preferably less than 20 μL/min, more preferably less than about 15 μL/min, more preferably less than about 12 μL/min, and more preferably less than about 10 μL/min.

In a preferred embodiment, CED comprises incremental increases in flow rate, referred to as “stepping”, during delivery. Preferably, stepping comprises infusion rates of between about 0.5 L/min and about 10 μL/min.

In a preferred embodiment, stepping comprises infusion rates of greater than about 0.5 μL/min, more preferably greater than about 0.7 μL/min, more preferably greater than about 1 μL/min, more preferably greater than about 1.2 μL/min, more preferably greater than about 1.5 μL/min, more preferably greater than about 1.7 μL/min, more preferably greater than about 2 μL/min, more preferably greater than about 2.2 μL/min, more preferably greater than about 2.5 μL/min, more preferably greater than about 2.7 μL/min, more preferably greater than about 3 μL/min, as well as preferably less than about 25 μL/min, more preferably less than 20 μL/min, more preferably less than about 15 μL/min, more preferably less than about 12 μL/min, and more preferably less than about 10 μL/min.

For further teaching on the method of CED, see for example Saito et al., Exp. Neurol., 196:381-389, 2005; Krauze et al., Exp. Neurol., 196:104-111, 2005; Krauze et al., Brain Res. Brain Res. Protocol., 16:20-26, 2005; U.S. Patent Application Publication No. 2006/0073101; and U.S. Pat. No. 5,720,720, each of which is incorporated herein by reference in its entirety. See also Noble et al., Cancer Res. Mar. 1, 2006;66(5):2801-6; Saito et al., J Neurosci Methods. Jun. 30, 2006;154(1-2):225-32; Hadaczek et al., Hum Gene Ther. March 2006;17(3):291-302; and Hadaczek et al., Mol Ther. July 2006;14(1):69-78, each of which is incorporated herein by reference in its entirety.

In a highly preferred embodiment, the method of CED is done with a CED-compatible reflux-free step design cannula. Such highly preferred cannulas are disclosed in Krauze et al., J Neurosurg. November 2005;103(5):923-9, incorporated herein by reference in its entirety, and in U.S. Patent Application Publication No. US 2006/0135945 A1, incorporated herein by reference in its entirety, and U.S. Patent Application Publication No. US 2007/0088295 A1, incorporated herein by reference in its entirety.

The present methods of treatment preferably involve one or more pre-operative diagnostic determinations of the presence or risk of a CNS disorder. Many biomarkers associated with various CNS disorders are known. For example, see Henley et al., Curr. Opin. Neurol., 18:698-705, 2005, incorporated herein by reference in its entirety. The diagnostic determination done preferably includes neuroimaging. The methods also preferably involve pre-operative imaging to stereotactically define the location of the targeted neuronal population. In one embodiment, the diagnostic determination involves a genetic test.

In a highly preferred embodiment, the methods additionally comprise imaging during administration in order to monitor cannula positioning. In one embodiment, the method comprises use of a neuronavigation system, for example, see U.S. Patent Application Publication No. 2002/0095081, incorporated herein by reference in its entirety.

In a preferred embodiment, the methods additionally comprise neuroimaging to monitor infusate distribution.

In one aspect, the invention provides methods of compiling data obtained from image-based monitoring of infusate distribution as delivered by CED to patients having a CNS disorder. The data may include but is not limited to volume of infusate, volume of distribution, neuroanatomical distribution, tumor volume and neuroanatomical location, tumor type, genetic data, tumor stage, tumor imaging data, infusion parameters, cannula parameters, and cannula placement data. In one embodiment the invention provides a database comprising such data. In one embodiment, the database is useful for deriving algorithms describing the distribution of infusate in the CNS of a patient having a CNS disorder and may be used to model therapeutic delivery.

It is contemplated that combinations of high molecular weight neurotherapeutics are used in methods herein. It is also contemplated that the high molecular weight neurotherapeutic be administered with an effective amount of a second therapeutic agent.

As disclosed herein, CED-delivered infusate is distributed, in part, through the perivascular space. Means for modulating heart rate and/or blood pressure are contemplated for use in the invention to modulate transport of infusate through the perivascular space. For a description of transport in the perivascular space of large mammals, see Krauze et al., Exp Neurol. November 2005;196(1):104-11, incorporated herein by reference in its entirety. With respect to perivascular space in rodents, see Hadaczek et al., Mol Ther. July 2006;14(1):69-78, incorporated herein by reference in its entirety.

Active agents include therapeutic proteins. Therapeutic proteins include biologically active variants. The active agents according to this invention may be isolated or generated by any means known to those skilled in the art. The term “variant” as used herein includes polypeptides in which amino acids have been deleted from (“deletion variants”), inserted into (“addition variants”), or substituted for (“substitution variants”), residues within the amino acid sequence of naturally-occurring active agent. Such variants are prepared by introducing appropriate nucleotide changes into the DNA encoding the polypeptide or by in vitro chemical synthesis of the desired polypeptide. It will be appreciated by those skilled in the art that many combinations of deletions, insertions, and substitutions can be made provided that the final molecule is biologically active.

The term “biologically active” as used herein means that the fragment of variant demonstrates similar properties, but not necessarily all of the same properties, and not necessarily to the same degree, as the active agent on which it is based.

The distance from the infusion site that a high molecular weight neurotherapeutic achieves varies with the parameters and agents used. Typically, the distance will be from about 1 mm to about 10 cm, though greater distances may be achieved (particularly with brain tumors, and subcortical diseases, esp. diseases of the midbrain and brainstem).

Pharmaceutical Compositions

Pharmaceutical compositions of the invention comprise a therapeutically effective amount of a high molecular weight neurotherapeutic in admixture with one or more pharmaceutically and physiologically acceptable formulation materials. For example, a suitable vehicle may be water for injection, physiological saline solution, or artificial CSF.

Once the pharmaceutical composition has been formulated, it may be stored in sterile vials as a solution, suspension, gel, emulsion, solid, or dehydrated or lyophilized powder. Such formulations may be stored either in a ready to use form or in a form, e.g. lyophilized, requiring reconstitution prior to administration.

The optimal pharmaceutical formulation will be determined by one skilled in the art. See for example, Remington's Pharmaceutical Sciences, 18th Ed. (1990, Mack Publishing Co., Easton, Pa. 18042) pages 1435-1712, incorporated herein by reference in its entirety. The final dosage regimen involved in a method for treating the above-described conditions will be determined by the attending physician, considering various factors which modify the action of drugs, e.g. the age, condition, body weight, sex and diet of the patient, the severity of any infection, time of administration and other clinical factors. As studies are conducted, further information will emerge regarding the appropriate dosage levels for the treatment of various diseases and conditions. As discussed above, the Vᵢ:V_{d} ratio varies between CNS regions, and Vᵢ will be adjusted accordingly without undue experimentation.

The pharmaceutical composition can typically include an effective amount of the respective high molecular weight neurotherapeutic in combination with a pharmaceutically acceptable carrier and, in addition, may include other medicinal agents, pharmaceutical agents, carriers, adjuvants, diluents, etc. By “pharmaceutically acceptable” is meant a material that is not biologically or otherwise undesirable, i.e., the material may be administered to an individual along with the selected agent without causing any undesirable biological effects or interacting in a deleterious manner with any of the other components of the pharmaceutical composition in which it is contained.

In a preferred embodiment, a pharmaceutical composition of the invention is locally deliverable into the CNS of a subject by CED.

In a preferred embodiment, the pharmaceutical composition comprises a tracing agent.

In a preferred embodiment, the tracing agent comprises an MRI magnet that may be used in conjunction with MRI to monitor distribution of infused pharmaceutical composition.

In a preferred embodiment, the MRI magnet is gadolinium chelate.

In a preferred embodiment, the tracing agent comprises a liposome, which comprises an MRI magnet. In a preferred embodiment, the MRI magnet is gadolinium chelate.

In one embodiment, the pharmaceutical composition comprises a facilitating agent. A facilitating agent is capable of further facilitating the delivery of active agent of a high molecular weight neurotherapeutic to target tissue. In a preferred embodiment, a facilitating agent is a biomolecule that is efficiently cleared from tissue. In a preferred embodiment, a facilitating agent has a short half life relative to an active agent. In a preferred embodiment, a facilitating agent is capable of competing with an active agent for binding to active agent binding sites in brain parenchyma. For additional description of facilitating agents, see U.S. Patent Application Publication No. 2002/0114780, incorporated herein by reference in its entirety.

An especially preferred facilitating agent for use in the present invention is low molecular weight heparin. Low molecular weight heparin (LMW Hep) has a broad therapeutic window and is safer than high molecular weight heparin (which may cause hemorrhage at same dose). High molecular weight heparin is an unfractionated form with a molecular weight range of 5,000 0-35,000 daltons.

The desired infusion volume, desired amount of active agent, and duration of infusion are largely determined by target tissue volume and the type of agent used, and are readily determined by one of skill in the art without undue experimentation.

Delivery Devices

In one aspect, the invention provides a delivery device comprising a pump that is capable of delivering a pharmaceutical composition of the invention by CED. The device comprises, or is used in conjunction with a catheter or cannula that facilitates localized delivery to a CNS population. Preferably a CED-compatible, reflux-free step-design cannula that is compatible with chronic or acute administration is used. In a preferred embodiment, the device further comprises a pharmaceutical composition of the invention.

Any convection enhanced delivery device may be appropriate for use. In a preferred embodiment, the device is an osmotic pump or an infusion pump. Both osmotic and infusion pumps are commercially available from a variety of suppliers, for example Alzet Corporation, Hamilton Corporation, Alza, Inc., Palo Alto, Calif.).

The catheter or cannula is inserted into CNS tissue in the chosen subject. One of skill in the art could readily determine which general area of the CNS is an appropriate target. Stereotactic maps and positioning devices are available, for example from ASI Instruments, Warren, Mich. Positioning is preferably conducted by using anatomical maps obtained by CT and/or MRI imaging of the subject's brain to help guide the injection device to the chosen target.

Kits

In one aspect, the invention provides kits for the treatment of CNS disorders, which kits comprise one or more pharmaceutical compositions of the invention. In one embodiment, a kit of the invention further comprises a delivery device useful for CED, preferably a cannula, and more preferably a step-design reflux-free cannula. In one embodiment, a kit of the invention further comprises a pump useful for CED. Kits may additionally comprise connecting parts, tubing, packaging material, instruction pamphlets, and other materials useful for practicing CED of a high molecular weight neurotherapeutic to the CNS of a patient having a CNS disorder.

Treatment of CNS Disorders

Treatment generally results in reducing or preventing the severity or symptoms of the CNS disorder in the subject, i.e., an improvement in the subject's condition or a “therapeutic effect.” Therefore, treatment can reduce the severity or prevent one or more symptoms of the CNS disorder, inhibit progression or worsening of the CNS disorder, and in some instances, reverse the CNS disorder.

As used herein, the term “ameliorate” means an improvement in the subject's condition, a reduction in the severity of the condition, or an inhibition of progression or worsening of the condition.

In the case of an acute CNS disorder, treatment will improve the subject's condition to a clinical endpoint, which may be amelioration of the disorder, complete or partial recovery from the disorder, at which point administration of high molecular weight neurotherapeutic is preferably discontinued.

An acute CNS disorder is one that may be effectively treated with administration of high molecular weight neurotherapeutic such that the subject's condition improves to a clinical point where administration may be discontinued. Examples of acute CNS disorders may include stroke and CNS trauma, though depending on severity, stroke and trauma may be considered chronic CNS disorders in need of chronic treatment.

The methods of the invention for treating a subject can be supplemented with other forms of therapy. Supplementary therapies include drug treatment, a change in diet, etc. Supplementary therapies can be administered prior to, contemporaneously with or following the invention methods of treatment. The skilled artisan can readily ascertain therapies that may be used in a regimen in combination with the treatment methods of the invention.

The specific dose is typically calculated according to the predetermined tissue distribution volume. The calculations necessary to determine the appropriate dosage for treatment involving pharmaceutical formulations is routinely made by those of ordinary skill in the art and is within the ambit of tasks routinely performed by them without undue experimentation.

All citations are expressly incorporated herein in their entirety by reference.

### EXAMPLES

Previous reports have shown that liposomes (Saito et al., Cancer Res. Apr. 1, 2004;64(7):2572-9, incorporated herein by reference in its entirety) and viral vectors (Chen et al., J. Neurosurg. 103:311-319, 2005, incorporated herein by reference in its entirety) may be interstitially infused into the rodent CNS. The robust distribution of liposomes obtained in small rodent brains does not guarantee similar results in the much larger primate CNS, and the relevance of these findings to clinical applications of CED is not clear given the pronounced physical and neuroanatomical differences between the normal CNS of rodents and humans, as well as the experimental infusion parameters used. Infusion into the rodent brain requires only a small volume of infusate for distribution in a small tissue volume which is achieved over a short period of time. Moreover, clinical application typically requires convection of therapeutics into pathologic tissue, e.g., tumor tissue, which is frequently heterogenous and differs markedly from normal brain tissue due to, for example, greater tissue density, high degree of vascularization and heterogeneous cytoarchitecture. Experiments were undertaken in primates to determine the feasibility and efficacy of high molecular weight neurotherapeutic delivery by CED to the CNS of large mammals, including large mammals with naturally occurring tumors.

### Example 1

### Gadolinium-Loaded Liposomes Allow for Real-Time Magnetic Resonance Imaging of Convection-Enhanced Delivery in the Primate Brain

The robust distribution of liposomes obtained in small rodent brains in the prior art did not guarantee similar results in the much larger primate CNS. Therefore, several key factors were further explored in the present studies. These included correlating the volume of infusion and volume of distribution in larger brains, developing a real-time imaging system, establishing the convectabilty of high molecular weight therapeutics in large mammal brain, and evaluating the accuracy of MRI monitoring for detection of liposome distribution.

Distribution of liposomes after convection-enhanced delivery detected by fluorescence labeling (data not shown): In order to test the feasibility of CED of liposomes in the non-human primate brain, liposomes (20 mM phospholipids) loaded with fluorescent dyes (either rhodamine or Dil-DS, the difference in fluorescent dye had no impact on liposomal distribution) were infused by CED at a volume of either 33 μl or 99 μl into the corona radiata or putamen of both hemispheres in 3 non-human primates. The animals were euthanized immediately after infusion. A robust distribution of liposomes was achieved and was detected at necropsy. Six data points were acquired [n=4 for the 33 μl infusions (3 corona radiata and 1 putamen) and n=2 for the 99 μl infusion (2 putamen)] out of 12 attempted infusion sites [2 infusion sites (corona radiata and putamen) per hemisphere in 3 monkeys]. The distribution volume was calculated and plotted against the infused volume. Two additional data points for a 99 μl infusion were added from the histological evaluation of the monkey brain used for the real-time MR monitoring study. Data from our previous study using CED of infusion volumes (Vi) of 5, 10, 20, and 40 μl in the rodent brain were plotted together with the primate data, and the correlation between Vi and Vd was calculated. A linear trend line detected a strong correlation between Vi and Vd (R²=0.95). According to these findings, a Vd twice as large as the Vi would be expected with Vi ranging from 5 to 99 μl. During real-time MR monitoring, we evaluated the volume of distribution of a 113.5 μl infusion (Vi) in two infusion sites in 1 monkey. The calculated volume (Vd) using histological sections (289 mm³ and 260 mm³) still stayed on the linear trend line, suggesting the accuracy of this acquired trend line.

Detection of liposomal gadolinium after convection-enhanced delivery in primates, and evaluation of toxicity (data not shown): To assess the feasibility of using MRI to monitor liposome distribution in the brain of non-human primates during CED and to confirm the safety of CED for drug distribution, CED of liposomal Gd was performed in 2 non-human primates. Targeted infusion sites were the right corona radiata, the left putamen, and the left brain stem. Infusion volumes consisted of 99 μl or 113.5 μl for the corona radiata and putamen, and 66 μl for the brain stem. Robust and clearly delineable distributions of liposomal Gd were observed at each infusion site in the T1-weighted MR images obtained immediately after infusion. MR images acquired 48 hours after infusion revealed retention of the liposomes at each infusion site with no adverse effects. The animals proceeded to a second infusion approximately 3 months later and were euthanized immediately following the second infusion. The animals developed no abnormal symptoms during the observation period. Postmortem hematoxylin and eosin staining showed some tissue damage due to the cannula. This damage was likely a consequence of long-term cannula placement, as the outer “guide” cannula was placed at the infusion site for more than 3 months. However, the tissue damage was limited to the cannula track despite the large distribution of liposomes. Throughout the study, there were no adverse clinical effects observed in any of the animals at any time points following CED of liposomal Gd.

Real-time magnetic resonance imaging of liposomal gadolinium in primates (data not shown): Real-time monitoring of liposome distribution was obtained in the corona radiata, putamen, and brain stem. MR images were obtained at approximately 10-minute intervals during the infusions. These real-time images detected liposomal distribution from approximately 10 to 20 minutes after initiation of infusion and clearly detected the enlargement of distribution area during infusion. The distribution areas were all well delineated from non-distributed brain tissue, again suggesting the feasibility of this strategy. During the brain-stem infusion of one non-human primate, unsuccessful infusion was also monitored with our real-time system, with too-close placement of the infusion cannula to the fourth ventricle, and no clear distribution was detectable throughout the procedure at the infusion site. However, in images more caudal to the targeted site, Gd signal was detected in the cerebrospinal fluid (CSF), suggesting a leak of infusate into the CSF.

Confirmation of distribution volume by histological detection using fluorescence (data not shown): Both animals used for real-time MR imaging of liposomal gadolinium received concomitant administration of fluorescent liposomes. One animal received real-time infusion of 99 μl of a liposomal mixture (liposomal Gd and rhodamine liposome) into the corona radiata and putamen and 66 μl into the brain stem, was euthanized immediately following MR imaging, and was processed for histological detection of fluorescence generated from the rhodamine liposomes that were co-infused with liposomal Gd. When compared with the MR image, the fluorescent area completely overlapped with the liposome distribution detected by MRI. Volume calculations performed with MRI and histological fluorescence data were 259 mm³ and 240.7 mm³ for the corona radiata, 210 mm³ and 223.5 mm³ for the putamen, and 83 mm³ and 77.8 mm³ for brain stem, respectively, which further confirmed that real-time MRI gives an accurate measurement of distribution volume. The second animal received real-time infusion of 113.5 μl of the liposomal mixture in both hemispheres, and was euthanized and processed in the same manner. Volume of distribution calculations performed with MRI and histological fluorescence data were 305 mm³ and 289 mm³ for the left hemisphere and 290 mm³ and 259.8 mm³ for the right hemisphere, respectively.

Robust distribution of gadoteriodol-loaded liposomes and three-dimensional real-time magnetic resonance imaging monitoring (data not shown): When volume of infusion (Vi) reached 300 μl, some leakage of the liposomal Gd was found at subarachnoid space. Thus the infusion was stopped at this point. From which site this leakage happened was not clear, however robust distribution was found at all three sites and almost entire striatum was covered with liposomal Gd at this point. Therefore this volume of infusion might be the maximum for the monkey brain. Volume of distribution was plotted against time elapsed from initiation of infusion. It took 141 minutes and 40 seconds to infuse total volume of 300 μl. Three-dimensional reconstruction of acquired images were also performed at the end of infusion.

For materials and methods and further discussion see Saito et al., Exp Neurol. December 2005;196(2):381-9, incorporated herein by reference in its entirety.

### Example 2

### Real-Time Visualization and Characterization of Liposomal Delivery into the Monkey Brain by Magnetic Resonance Imaging

Magnet resonance imaging of Gadoteridol-loaded liposomes during CED in primate brain (data not shown): Infusion was started simultaneously in all three targeted regions (brainstem, putamen and corona radiata) of primate CNS, and placement of cannulas was verified before infusion pumps were turned on. After stabilization of animal vital signs, up to 700 μl of liposomes was convected with increasing rates of infusion. Robust and reflux-free Vd was achieved at all 3 sites. Brainstem infusion distributed rostrally towards mid-brain and caudal towards medulla oblongata. Some distribution into cerebellum via superior cerebellar peduncle was seen at 700 μl infusion. Liposomal distribution in corona radiata was primarily confined to white matter, and distributed into the non-infused contralateral hemisphere via corpus callosum above 500 μl infusion volume. Infusion in putamen was well contained at infusion volumes less than 300 μl. Beyond 300 μl, distribution was seen to expand further in anterior and posterior directions within the putamen. However, in coronal views, the signal was seen to distribute beyond the lateral borders of the putamen into internal and external capsules. Signal was also detected in perivascular space of middle cerebral artery after infusion of 100 μl liposome.

Three-dimensional (3D) reconstruction of a 700-μl liposomal infusion in primate CNS: Liposomal signal seen on MRI was outlined with BrainLab software, and a 3D reconstruction of Vd was obtained. A sagittal view, with digital subtraction at midline, was used to visualize distribution in pontine brainstem and corona radiata. The MR image shows the structure-related volume of distribution of liposomes with almost complete perfusion of brainstem and robust distribution along white matter tracts of corona radiata.

Volume of distribution (Vd) was calculated and plotted against volume of infusion (Vi). BrainLab software was used to determine Vd from MRI, and NIH Image was used to analyze and delineate histology sections, and calculate Vd. Results of all three infusion sites show a linear correlation of Vi and Vd with the following equations: putamen: y=0.0009x+0.0487, R²=0.9654; corona radiata: y=0.0014x+0.0779, R²=0.9531; brainstem: y=0.002x+0.1466, R²=0.9753. The lowest Vd of 0.684 cm³, after infusion of 700 μl liposomes, was seen in putamen, followed by corona radiata with about 1 cm³ after a Vi of 700 ml liposomes. Maximum distribution was seen in brainstem, yielding around 1.6 cm³ for 700 μl Vi. The distribution ratio at 700 μl (Vd/Vi) was as follows: 97.7% for putamen, 142.8% for corona radiata and 228.5% for brain stem. The R² values in show a linear correlation of each CNS structure with respect to increasing infusion volume.

Liposome distribution on primate histology sections (data not shown): Almost entire coverage of the brainstem was achieved after a 700 μl liposome infusion. Infusion into Putamen shows the smallest distribution within all structures infused of the primate CNS. Distribution, mainly along white matter fiber tracts, is seen at Corona Radiata infusion side. As already seen on MR images, liposomal distribution at the Corona Radiata infusion site crosses over to the contralateral hemisphere via the white matter tracts of the corpus callosum.

For materials and methods and further discussion see Krauze et al., Brain Res Brain Res Protoc. December 2005;16(1-3):20-6, incorporated herein by reference in its entirety.

### Example 3

### Effects of the Perivascular Space on Convection-Enhanced Delivery of Liposomes in Primate Putamen

MRI monitored leakage out of non-human primate striatum after liposomal infusion (data not shown): We established a method to monitor in real time the infusion of liposomes loaded with a surrogate marker. We then used this system to infuse various anatomical structures in non-human primate brain including putamen. CED of up to 300 μl of liposomes was performed in non-human primate putamen, and subsequent distribution was monitored. Placement of cannula in primate putamen was verified for each animal by MRI prior infusion of liposomes. MRI was used to monitor CED of liposomes throughout the infusion procedure and reflux-free delivery was established to ensure optimal convection parameters. After starting the primate putamen infusion procedure, signal enhancement was detected in the perivascular space of the medial cerebral artery (MCA). At the lateral putamen border, lateral striate arteries (LSA) also showed signal enhancement. Volume infused into each animal at which MCA signal enhancement was first seen on MRI was as follows: #A-50 μl, #B-20 μl, and #C-15 μl. Signal enhancement continued to spread in the perivascular space along branches of MCA. Increasing signal enhancement in the Sylvian fissure and insular region was also visible, while infusion of liposomes into putamen continued with perivascular MCA signal present. No signal in the external capsula bordering on insular cortex was seen throughout the infusions.

MRA (Magnetic Resonance Angiography) of non-human primate cerebral vessels (data not shown): The signal seen in primate cerebral arteries after performing MRA shows the luminal MCA signal in coronal, axial and sagittal views. This signal location exactly matched liposomal MRI signal seen after putamen infusions in same anatomical views. Results of this study confirmed the (perivascular) arterial origin and perivascular transport of the liposomal signal seen during intra-putaminal infusions. Post-mortem examination confirmed localization of LSA with respect to perivascular transport of liposomes seen during MRI.

Reconstruction in three Dimensions of putamen infusion and leakage pathway: To understand special relationship between localization of the vessels and the pattern of perivascular transport of liposomes 3-dimensional (3-D) reconstruction was performed. Although MCA leakage was seen first after 50 μl infusion (Animal #A), data for delineation was taken at the 150 μl infusion volume in order to visualize the complete leakage pathway. This reconstruction enabled us to demonstrate clearly distribution in the putamen, and leakage of liposomes, in relation to MRI data. Digital subtraction of MR image allowed further detailed analysis of the leakage pathway in relation to primate brain anatomy. Insula and Sylvian fissure again display accumulation of liposomal signal seen in MR imaging during the infusion procedure.

Analysis of anatomical structures and fluorescence along the leakage pathway (data not shown): In order to correlate data obtained in-vivo with post-mortem examination MRI data were compared with data from histological analysis of non-human primate brains. Vessels of the lateral striate arteries with perforating branches, that are the arterial supply for the putamen, are seen at the lateral putamen border in a more ventral section seen from infusion site.

Co-infusion of gadoteridol- and sulforhodamine B-loaded liposomes allowed us to perform primate histology for fluorescent marker, thus making comparison between MRI and histological demonstration of perivacular transport possible. Minimal leakage followed by subsequent dilution and rapid clearance of the hydrophilic markers diminishes the signal contribution of extraliposomal gadoteridol and sulforhodamine B. Structures that were enhanced on MRI were histologically analyzed for fluorescence to confirm the perivascular origin of transport. A histological section that contained putamen, LSA vessels and MCA vessel were analyzed to demonstrate structures involved in leakage pathway. Clear fluorescent signal can be seen at the basal areas of brain surrounding the perivascular space of a MCA vessel.

For materials and methods and further discussion see Krauze et al., Exp Neurol. November 2005;196(1):104-11, incorporated herein by reference in its entirety.

### Example 4

### High Molecular Weight Neurotherapeutics are Convectible in Canine Brain Tumor Tissue and Promote Tumor Growth Arrest and Reduction

The canine brain tumor model is the best model of the human condition for the study of safety and distribution of locally administered therapeutics prior to clinical application. Tumors of the CNS occur more frequently in canines than in any other domestic species. The reported incidence of primary brain tumors in canines is 14.5 per 100,000—slightly higher than that reported in humans. Of the primary brain tumors, glial cell tumors (e.g., astrocytoma, oligodendroglioma, and mixed/poorly differentiated gliomas) are reported to be among the most common. Canine primary brain tumors exhibit remarkable similarities to their human counterparts in terms of histopathology, imaging characteristics, biologic behavior, and response to conventional treatment modalities. Similar to humans, the prognosis for dogs with primary brain tumors is poor. Even with available treatment regimens, including surgery, radiation therapy, and chemotherapy, reported survival times are rarely greater than 6 months for gliomas and 1 year for meningiomas. It is likely that the underlying molecular abnormalities resulting in human and canine tumors also will be similar in many cases.

Preliminary data looking at the expression of growth factors and their receptors, as well as interleukin 13 receptor 2α (IL13R2α), in canine primary brain tumors show striking similarity to data published for comparable human primary brain tumors. Vascular endothelial growth factor (VEGF) and its major receptors VEGFR-1 (fit-1) and VEGFR-2 (KDR) are primarily over-expressed in high-grade astrocytic and oligodendroglial tumors. A similar pattern of over-expression is seen with IL-13R2α. Platelet-derived growth factor receptor a (PDGFRα) is over-expressed in high-grade oligodendrogliomas, but not in meningiomas. Epidermal growth factor receptor (EGFR) is over-expressed predominantly in high-grade gliomas, but also in some lower-grade astrocytomas and meningiomas. All of these growth factors and receptors are thought to play significant roles in the pathogenesis of CNS tumors in humans and dogs. These data suggest that in addition to the similarities in histology, imaging, and biological behavior, canine primary brain tumors may have many of the molecular characteristics of their human counterparts, and provide a clinically valuable in vivo, spontaneous, large-animal model of human primary brain tumors.

For materials and methods concerning CED delivery of high molecular weight neurotherapeutics, see Krauze et al., Brain Res. Protocols 16:20-26, 2005, incorporated herein by reference in its entirety.

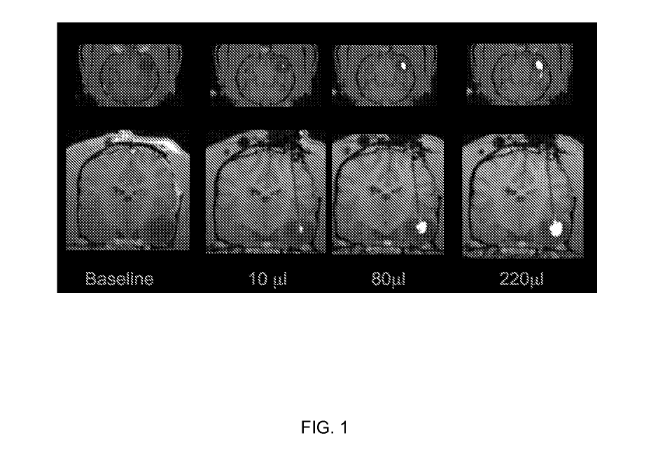

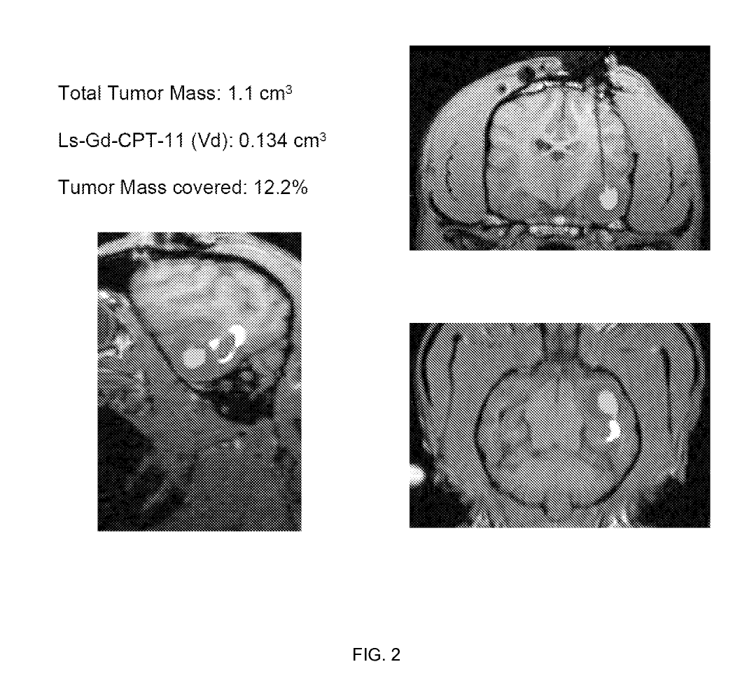

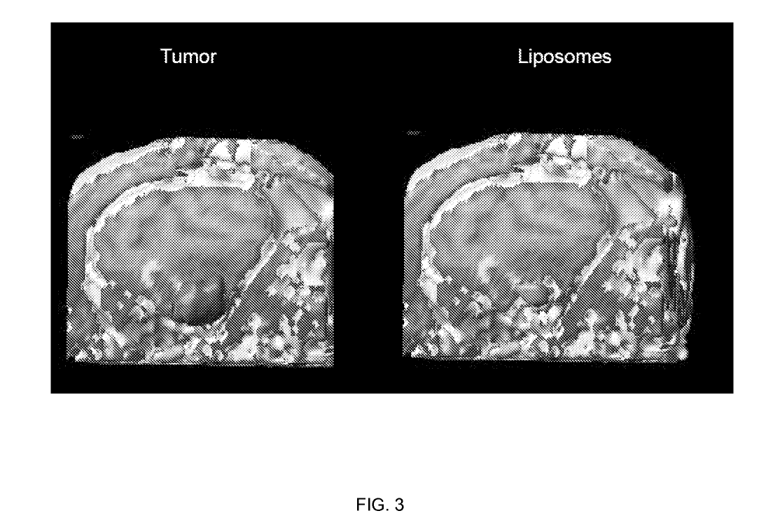

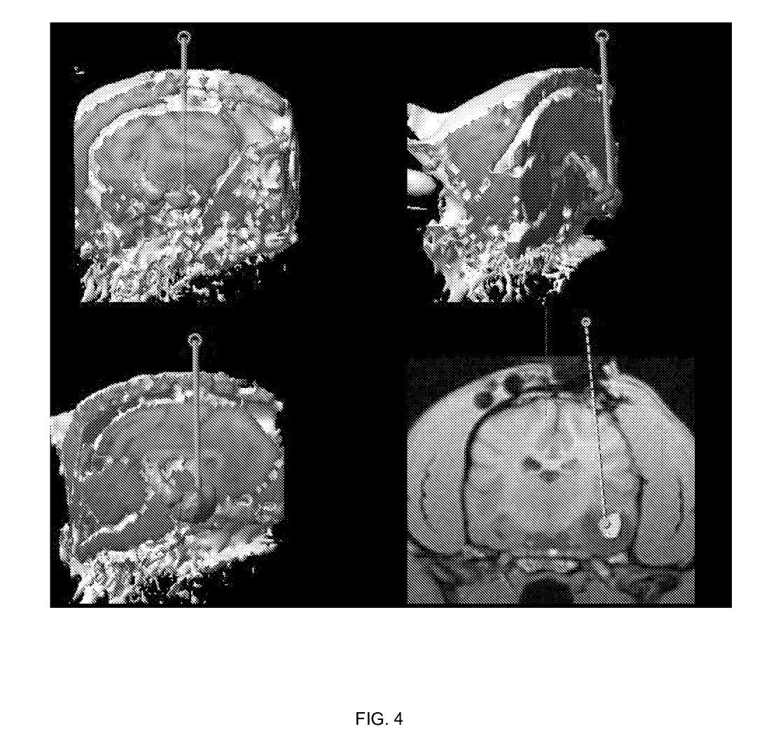

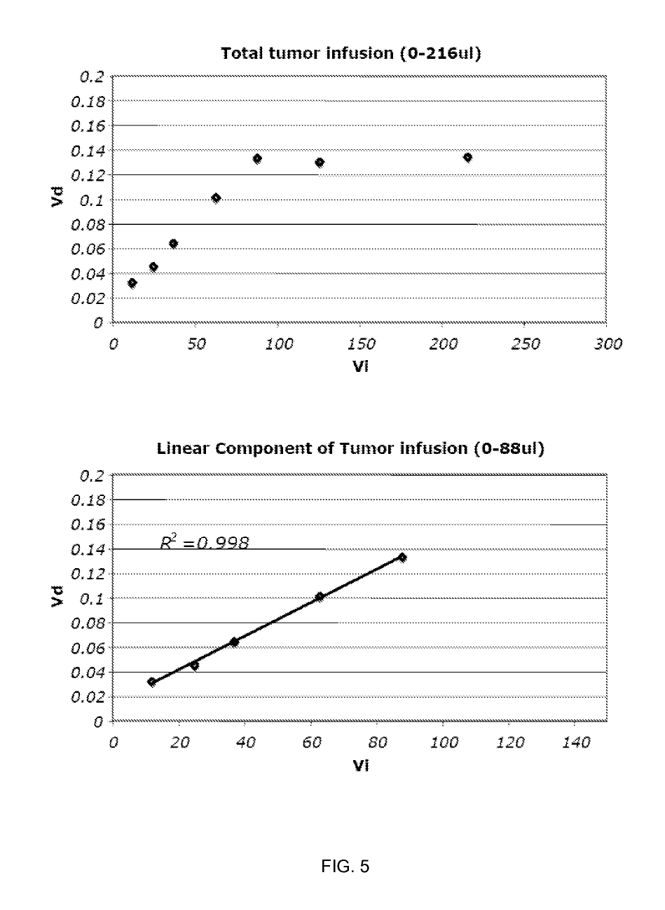

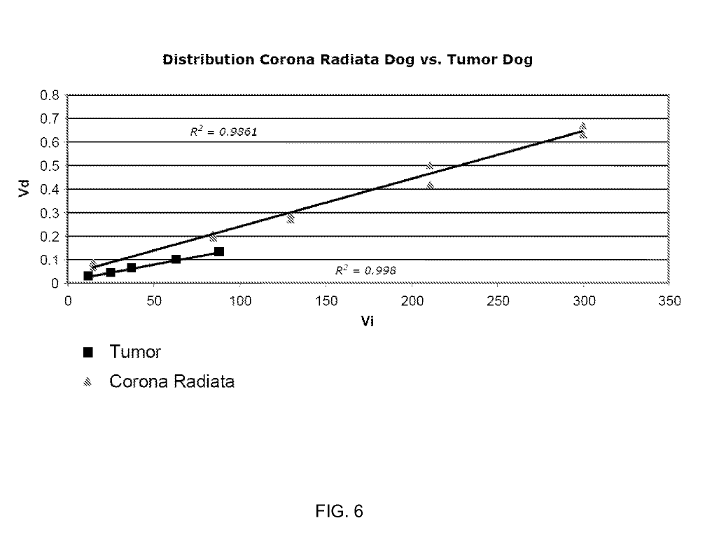

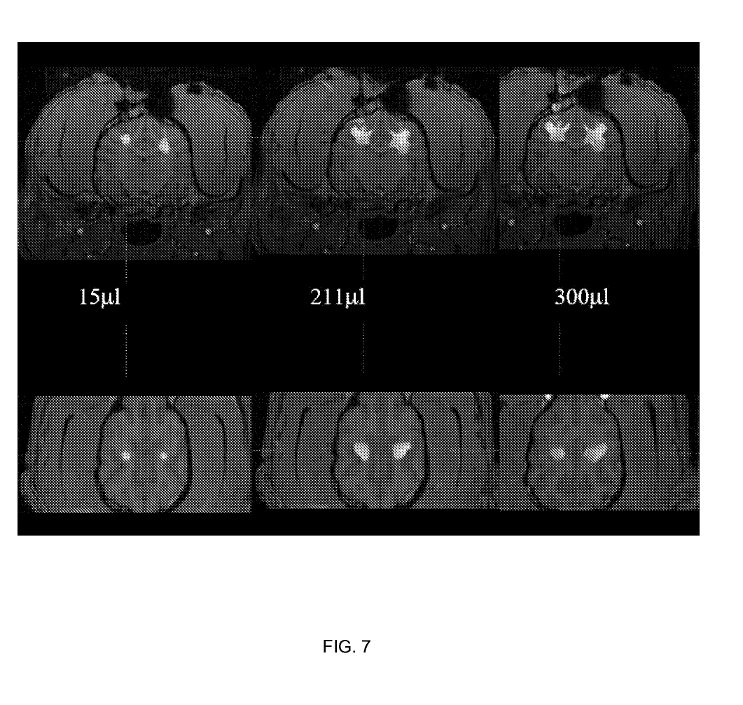

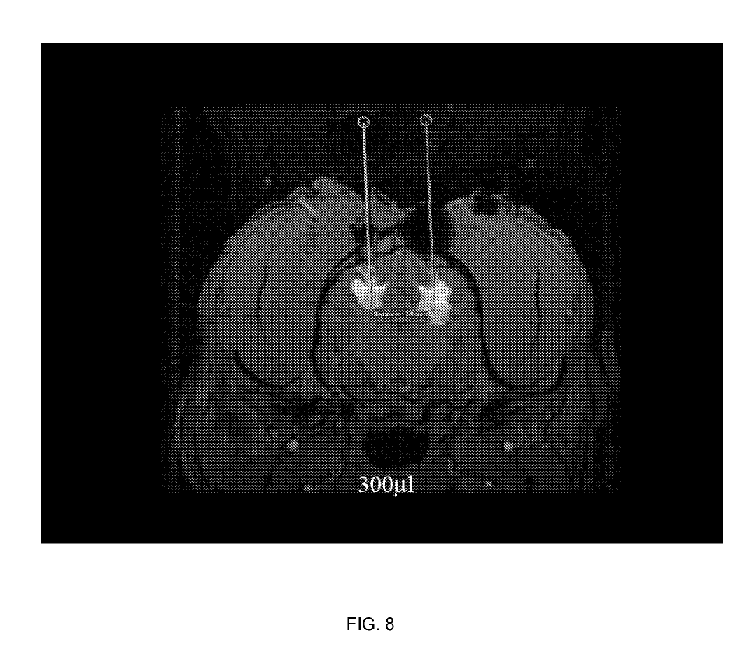

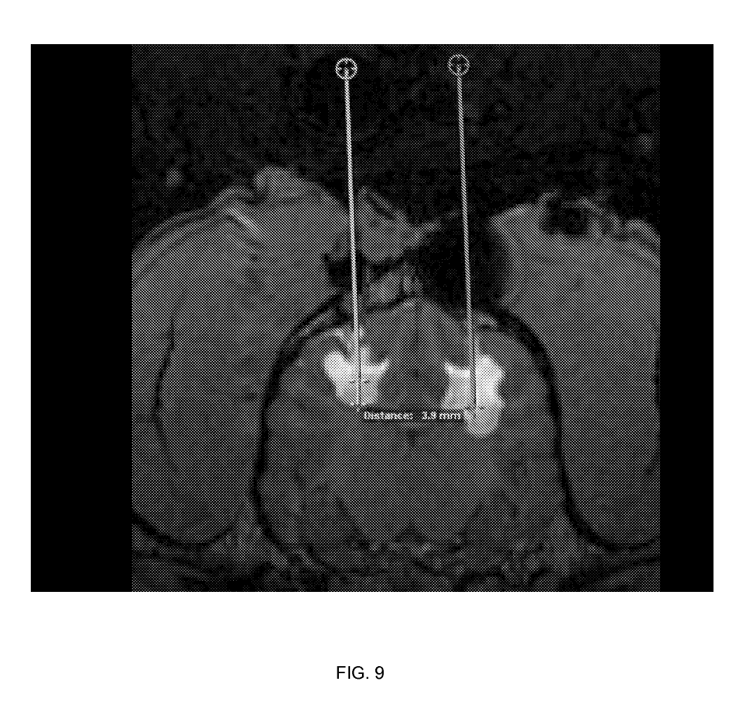

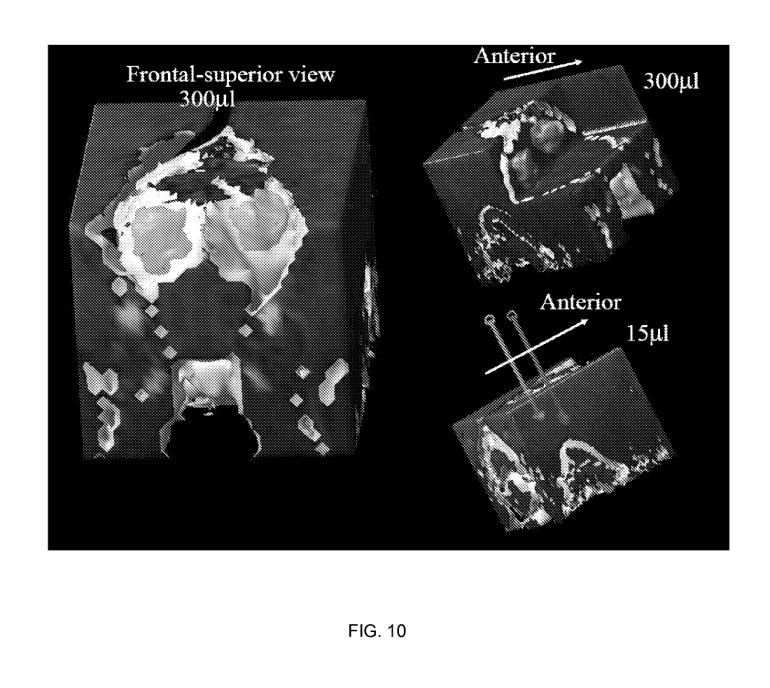

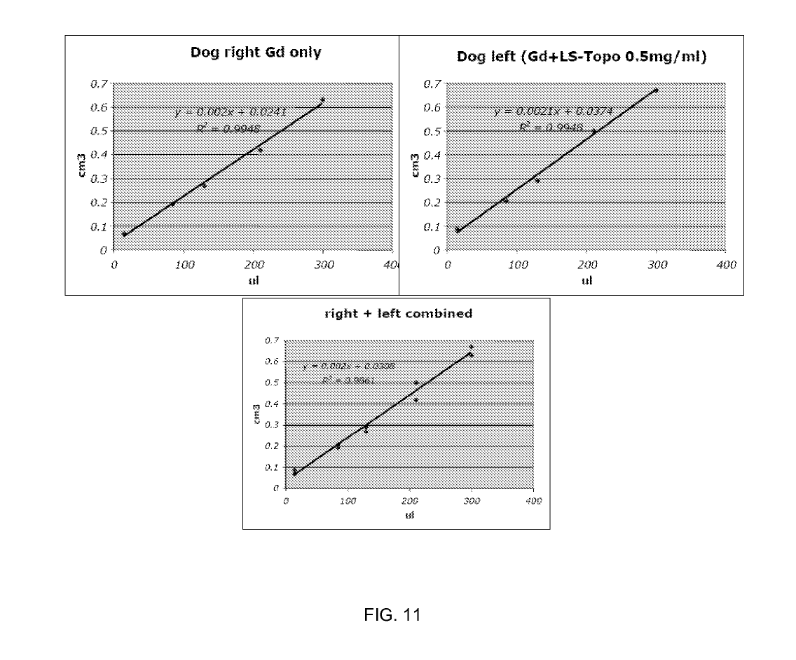

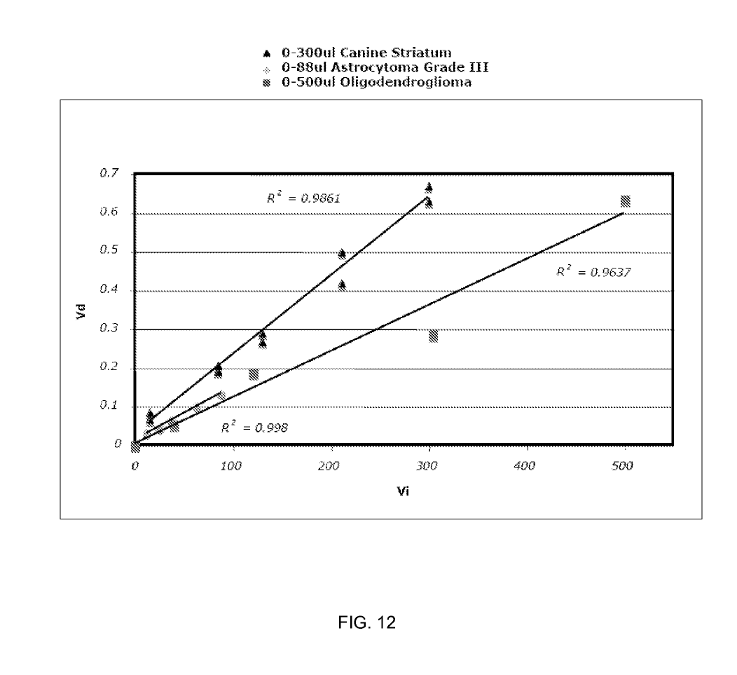

We administered combination CED of liposomal CPT-11 (Topoisomerase I inhibitor) and GDL in canine patients with brain tumors. Liposomal CPT-11/GDL infused by CED was monitored with real-time MRI, indicating accurate targeting of the brain tumor and providing us with data describing pattern of distribution of liposomes in brain tumors and efficacy of treatment. Three cases were studied and are presented below. Preliminary experiments were done in tumor dogs to establish distribution of liposomes within tumors monitored in real-time on MRI (FIG. 1-12).

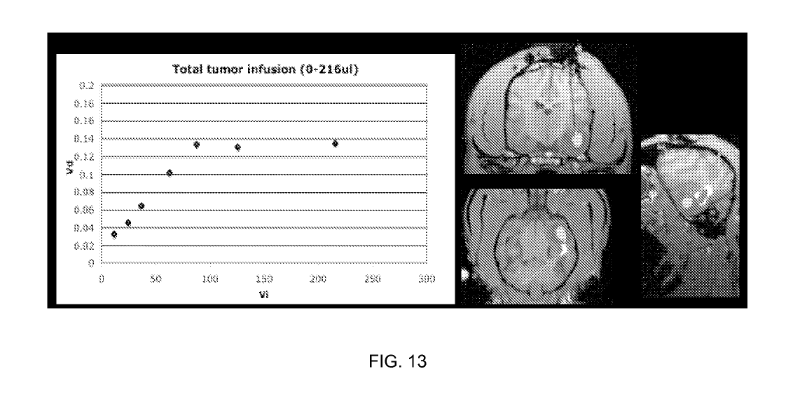

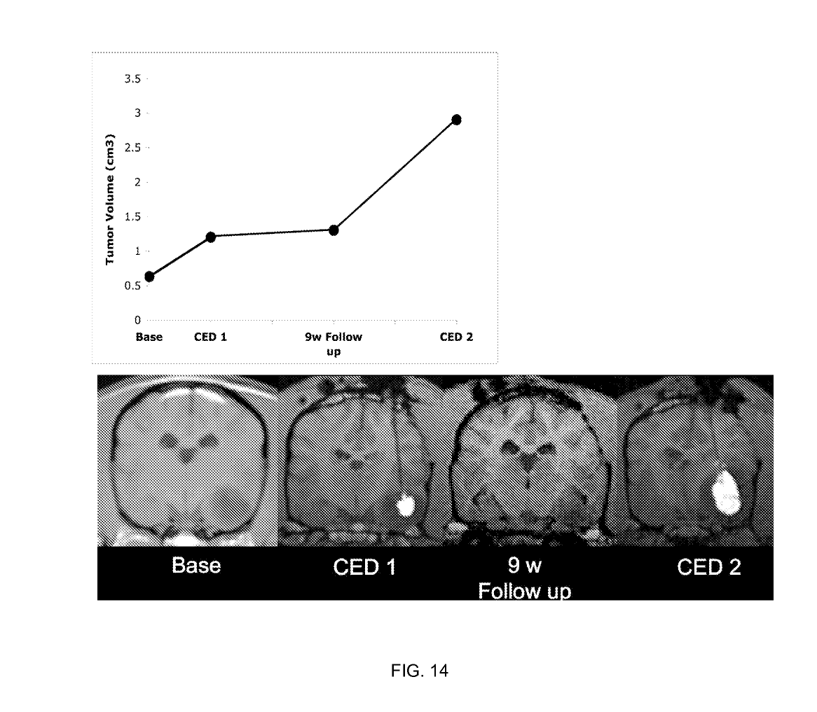

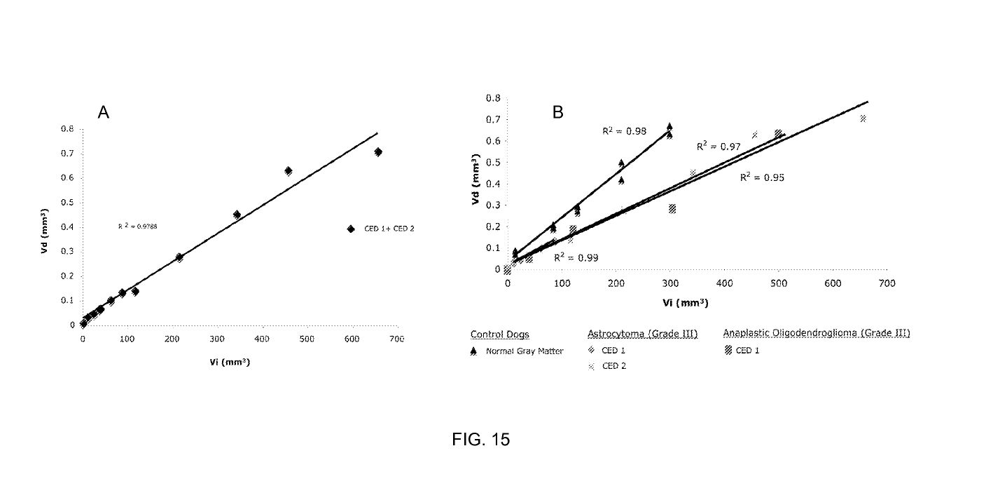

Case number 1: Brain biopsy was performed and dog was diagnosed with pyriform lobe grade III astrocytoma. Using real-time MRI (FIG. 13) mixture of CPT-11 and GDL (220 μl) was infused directly into the tumor over a 2.5 hr period at a maximum infusion rate of 3 μl/min. The volume of distribution was linear for the first 88 μl and then reached a plateau due to a leakage of infusate into the temporal horn of the lateral ventricle (arrow FIG. 13) as the expanding infusate border contacted the ventricular margin. This result underscores need for monitoring of local delivery of therapeutics, including liposomes, into the brain tumors. Use of GDL proved to be a very effective tool for monitoring distribution of CPT-11 liposomes in the canine brain tumor. Follow-up MRI, 9 weeks after the treatment revealed that there was significant reduction of tumor growth (FIG. 14) when compared with baseline, mostly in the region that was covered by the infusate. The arrest of tumor growth seen 9 weeks after CED-1 is consistent with presence of CPT-11 in the brain tumor. Note that we had only covered 12% of tumor during CED-1 treatment. FIG. 14 describes tumor volume at time of diagnosis (Base) at the time of first (CED-1) treatment, 9 weeks follow-up MRI scan and at second treatment (CED-2). Tumor growth is seen again beyond 9 weeks, and a second treatment by CED is done where large regions of tumor (˜25%) are covered. The second treatment significantly reduces tumor growth. We evaluated Vᵢ:V_{d} ratio obtained at both treatments and found that there is a very close correlation of Vᵢ to V_{d} regardless of the volume infused suggesting that we can distribute GDL in reproducible matter not only in the healthy brain tissue but also in brain tumors (FIG. 15). This shows the reproducibility of repeated CED in brain tumor tissue in a large animal model.

Histopathological evaluation of case number 1 following death unrelated to brain tumor. History: An 11 year old female neutered Jack Russell Terrier was diagnosed with a suspected glioma in the right piriform lobe based on T1/T2 weighted and post-contrast MR studies and confirmed histologically and immunocytochemically with GFAP from a CT-guided stereotactic biopsy as a diffuse fibrillary astrocytoma Grade II. The dog received three CED intratumoral infusions even spaced over 5 months with post infusion monitoring of tumor response by MR imaging. The dog was euthanized 8 months after the first treatment due to complications from a disseminated hemangiosarcoma which did not metastasize to the brain.

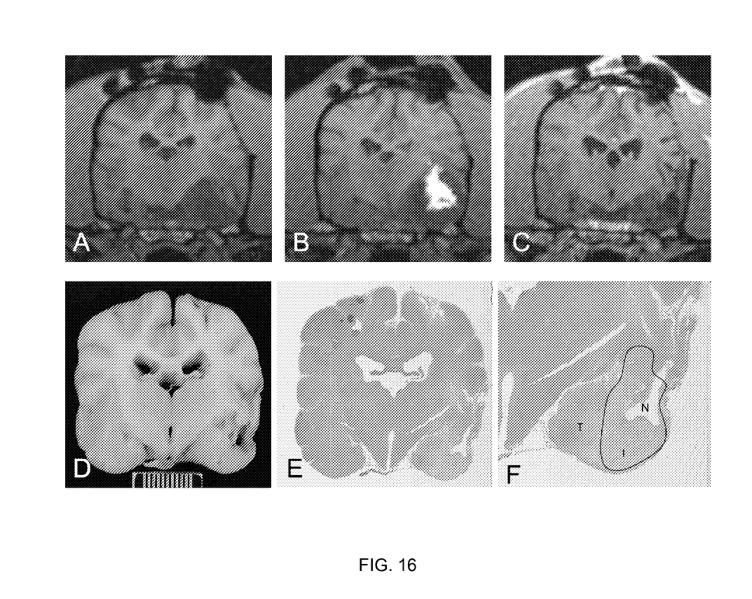

Neuropathology and conclusions: (FIG. 16) After necropsy, on transverse sections of the brain both grossly and microscopically through the area of the CED of the intra-tumoral infusion, containing CPT-11 in liposomes with gadolinium, as defined by MRI there was an area of malacia at the tip of the catheter (N) and an outer zone of a low grade diffuse modified low grade fibrillary astrocytoma (I). In the outer border of the preexisting tumor which was not infused (T) there was a diffuse astrocytoma Grade II. A major finding was that in the infused area of morphologically modified tumor there was a proliferation index as determined by MIB-1 immunocytochemistry of <1% compared with about 18% in the non-infused astrocytoma (data not shown). We conclude from this experiment that repeated intratumoral infusions of the liposome/CPT-11/gadolinium resulted in intratumoral necrosis and in profound CPT-11/liposomal-induced suppression of MIB-1 activity within the modified fibrillary astrocytoma compared with the adjacent non-infused high grade astrocytoma. These findings underscore the importance of drug distribution in brain tumor treatment.

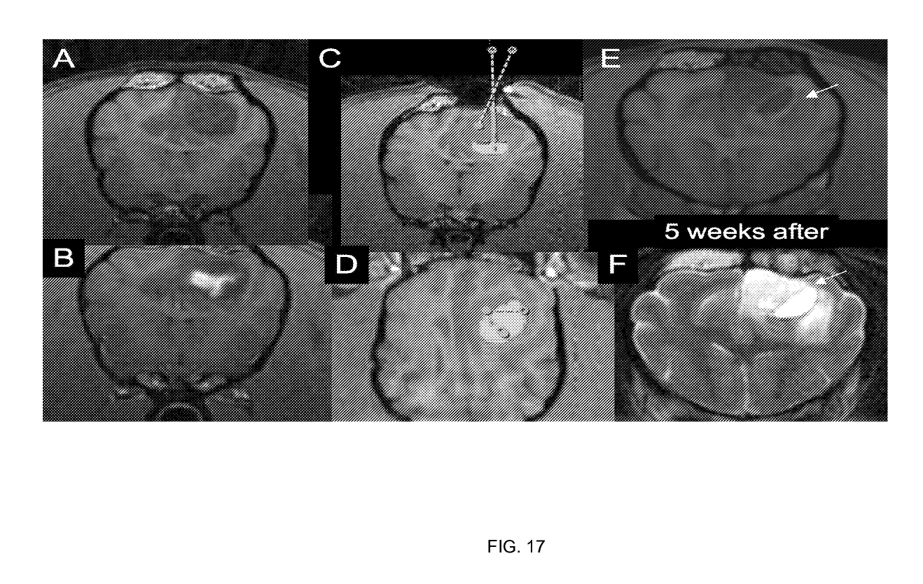

Case number 2: Biopsy in the second case confirmed frontal/parietal lobe anaplastic oligodendroglioma (grade III) (FIG. 17). In this case liposomal Gd (1.85 mM) and CPT-11 (48.2 mg/ml) were infused via a two cannulae placed into the rostral and caudal aspects of the tumor. A total volume of 500 μl over a 2.5 hr period with a maximum infusion rate of 3 μl/min. Rostral cannula placement was suboptimal and infusion via this catheter was minimal. Volume of distribution was essentially linear with a Vd:Vi ratio of approximately 1.33. Final volume was limited by available infusate and anesthetic limitations and a total of 18% tumor volume was infused. Distribution of infusate within the tumor was markedly different than for case 1, with the infusate following the border of T2 hyperintensity and peripheral margins of the tumor. Post-infusion MRI examination at 6 weeks revealed that tumor did not grow and was reduced in size in the region of drug administration. Some tumor ablation that correlates to CPT-11 infusion site was also observed on both T-1 and T-2 MRI (FIG. 16E,F).

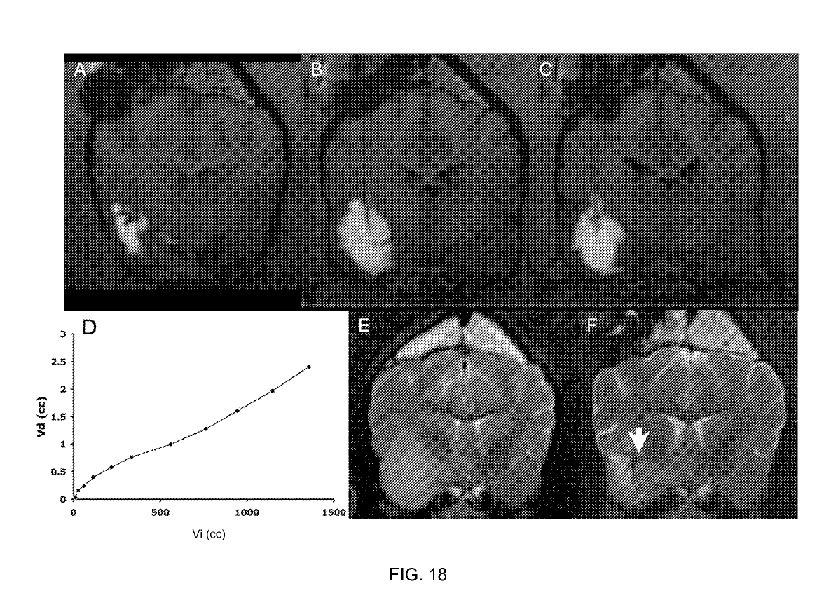

Case number 3: A brain biopsy confirmed the diagnosis of a pyriform lobe grade III astrocytoma. Dog presented with neurological signs including seizures. Guide cannules were placed over the tumor and 3 sites were targeted as shown in FIG. 18. Majority of the tumor was covered by the CPT-11/GDL using real-time MRI-guided CED. Infusion was stopped once small leakage at the base of the brain was detected at which time point almost whole tumor mass was treated. This patient dog remained symptom free for over 3 months and was followed with MRI every 6 weeks. MRI showed dramatic reduction in the tumor mass (FIG. 18), similarly to what had been in Case 1.

These results show that real time monitoring of liposomal therapeutics to normal brain and spontaneous gliomas is feasible and distribution is highly predictive, based on co-infusion of surrogate gadolinium markers. The adverse effects associated with the infusions appear to be minimal.

The current studies underline the importance of monitoring drug delivery to the CNS and demonstrate that direct infusion of therapeutic agents into spontaneous CNS tumor tissue is feasible and distribution (as measured by co-infusion of surrogate markers) is highly predictive of effectiveness. Adverse effects associated with the infusions appear to be minimal.

Real-time imaging of infusions is likely to be a critical, if not an essential component of CED if therapeutic efficacy is to be maximized, and toxicity associated with inappropriate cannula placement or leakage into peri-tumoral structures such as the ventricles is to be minimized.

Methods: Magnetic resonance imaging. MRI methods are as in primate studies, see Saito et al., Exp Neurol 196:381-9, 2005; Krauze et al., Exp Neurol 196:104-11, 2005; each of which is incorporated herein by reference in its entirety.

Surgical procedures for guide cannula implantation: Guide cannula preparation: In the surgery room, a sterile field was created to prepare each guide cannula for implantation. Briefly, a custom-designed guide cannula was previously prepared by inserting fused silica into pedestal screws (13 mm) and securing with superglue. On the day of surgery, the fused silica portion of the cannula was cut to a specified length (3-5 mm) to accommodate the needle trajectory for each target site. A corresponding nylon dummy cannula with stylet was cut to the same length to avoid tissue buildup within the system. The cannula was flushed with sterile saline and transferred to the surgery table. Guide cannula was prepared during surgery to accommodate targeted regions of the brain. In clinical animals, the location and number of catheters was determined based on baseline MRI findings obtained from the experimental studies.

Surgery procedures: Prior to induction of anesthesia, dogs were pre-medicated with oxymorphone (0.04-0.06 mg/kg, SC), diazepam (0.03-1.0 mg/kg IM), and atropine (0.02-0.04 mg/kg SC), induced with thiopental (10.0 mg/kg IV), and intubated. Anesthesia was maintained with isoflurane (1.5% in oxygen) and a PaCO₂ was maintained between 30-35 mmHg using positive pressure ventilation. Body temperature was maintained between 37.5-39.0° C. with the aid of circulating air/water blankets. An intravenous cephalic catheter, an indirect pressure cuff, and EKG leads were placed for monitoring of mean arterial blood pressure and EKG while under anesthesia. Blood gasses, blood glucose, and electrolytes were monitored every 30 to 60 minutes during anesthesia. Intravenous fluid administration (Lactated Ringer's solution, 10-12 ml/kg/hr) was continuous throughout the anesthetic period. Temperature, respiratory rate, heart rate, mucous membrane color, and mentation were monitored every 10 minutes during anesthetic recovery. When animals were fully recovered, a veterinary neurologist assessed neurological signs prior to returning the animal to the housing facility.

The dog's head was placed in a canine MRI compatible stereotactic frame prior to obtaining an initial baseline MRI that determined the location of the guide cannula assembly. Surgical exposure for placement of cannulae involved a midline skin incision and retraction of the temporalis muscle to expose the cranium over the cannula entry site. Using a Hall air drill, a small burr hole was made in the skull to expose the dura over the infusion site. A 21-gauge needle was used to penetrate the dura to expose the cortex above each infusion site and additional burr holes were created adjacent to each infusion site to position brass set screws. Using a stereotactic tower, each guide cannula assembly was stereotactically lowered into the burr hole, the hole filled with acrylic, and the cannula assembly secured using dental acrylic. Once the guide cannula was secured, additional acrylic was applied to bond the guide to several screws positioned on the skull. The wound site was closed in anatomical layers over the guide cannula. Each animal was monitored for full recovery from anesthesia, placed on antibiotics and observed twice daily for 5 days following surgery.

Liposome preparation: For example, see Noble et al., Cancer Res. Mar. 1, 2006;66(5):2801-6. 1,1′-dioctadecyl-3,3,3,3′-tetramethylindocarbocyanine-5,5′-disulfonic acid (DiIC₁₈(3)-DS) was obtained from Molecular Probes (Eugene, Oreg.), 1-2-dioleoyl-3-sn-glycerophospho-choline (DOPC) and poly(ethylene glycol)-1,2-distearoyl-3-sn-phosphoethanolamine (PEG-DSPE) from Avanti Polar Lipids (Alabaster, Ala.), and cholesterol (Chol) from Calbiochem (San Diego, Calif.). DOPC and Chol (molar ratio 3:2), PEG-DSPE (5 mol %) and optional DiIC₁₈(3)-DS (0.2 mol %) were mixed in chloroform and dried by rotary evaporation. For MRI studies, liposomes were passively loaded with Gd (Omniscan®) (GD-liposomes). The lipid film was rehydrated in Gd solution (250 mM), followed by 6 successive cycles of rapid freezing-thawing, and was subsequently extruded through polycarbonate filters with defined pore sizes (5×0.2 μm, 5×0.05 μm), yielding liposomes of ˜80 nm diameter as determined by dynamic light scattering. Unencapsulated Gd was removed using a Sephadex G-75 size exclusion column (Pharmacia, Piscataway, N.J.), followed by extensive dialysis against HEPES buffered saline (HBS) (pH 6.5). Liposome concentration was measured by standard phosphate analysis and adjusted to 20 mM phospholipid for all experiments.

The following publications are incorporated herein by reference in their entirety: Langer, “New methods of drug delivery,” Science, 249:1527-1533 (1990); Morrison, “Distribution models of drug kinetics,” in Principles of Clinical Pharmacology, Atkinson et al (eds), Academic Press, New York, pp 93-112 (2001); and Pardridge, “Drug delivery to the brain.” J Cereb Blood Flow Metab, 17:713-731 (1997); Krauze et al., Exp Neurol. November 2005;196(1):104-11); Saito et al., Cancer Res. Apr. 1, 2004;64(7):2572-9; Chen et al., J. Neurosurg. 103:311-319, 2005; Vandevelde et al., Acta Neuropathol (Berl) 66:111-6,1985; Koestner et al. Histological Classification of Tumors of the Nervous System of Domestic Animals. 2nd ed. Washington, D.C.: The Armed Forces Institute of Pathology; 1999; Gavin et al., J Neurooncol 33:71-80,1997; Kraft et al., J Vet Intern Med 11:218-25, 1997; Lipsitz et al., Vet Pathol 40:659-69, 2003; Thomas et al., Vet Radiol Ultrasound 37:20-27,1996; Axlund et al., J Am Vet Med Assoc 221:1597-600, 2002; Jeffery et al., J Small Anim Pract 34:367-372, 1993; Brearley et al., J Vet Intern Med 13:408-12, 1999; Spugnini et al., Vet Radiol Ultrasound 41:377-80, 2000; Dimski et al., J Am Anim Hosp Assoc 26:179-182, 1990.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art. In the appended claims, reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present disclosure and claims. Moreover, it is not necessary for a device or method to address each and every problem sought to be solved by the present invention, for it to be encompassed by the present disclosure and claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. A method of treating a patient having a central nervous system (CNS) disorder, comprising:
administering to said patient a therapeutically effective dose of a pharmaceutical composition comprising a high molecular weight neurotherapeutic by convection enhanced delivery (CED) to the CNS;
wherein said high molecular weight therapeutic comprises a carrier and an active agent.

2. The method according to claim 1, wherein said carrier is a synthetic carrier.

3. The method according to claim 2, wherein said synthetic carrier is a liposome.

4. The method according to claim 1, wherein said high molecular weight neurotherapeutic has a molecular weight greater than about 200 kDa.

5. The method according to claim 1, wherein said high molecular weight neurotherapeutic has a diameter or length greater than about 10 nm.

6. The method according to claim 1, wherein said high molecular weight neurotherapeutic comprises an active agent selected from the group consisting of nucleic acids, proteins, and small molecule chemical compounds.

7. The method according to claim 1, wherein said CED to the CNS is performed with a V_{d}:Vᵢ greater than 1:1.

8. The method according to claim 1, wherein said pharmaceutical composition further comprises a tracing agent, and said method further comprises monitoring distribution of said tracing agent.

9. The method according to claim 8, wherein said tracing agent is an MRI magnet, and said monitoring distribution of said tracing agent involves MRI.

10. The method according to claim 9, wherein said MRI magnet is gadolinium chelate.

11. The method according to claim 1, wherein said CED to the CNS is performed with a step-design reflux-free cannula.

12. The method according to claim 1, wherein said CNS disorder is an acute CNS disorder.

13. The method according to claim 1, wherein said CNS disorder is a chronic CNS disorder.

14. The method according to claim 1, wherein said CNS disorder is cancer.

15. The method according to claim 1, wherein said CNS disorder is a neurodegenerative disease.

16. The method according to claim 1, wherein said active agent is selected from the group consisting of antineoplastic agents, radioiodinated compounds, toxins (including protein toxins), cytostatic or cytolytic drugs, genetic and viral vectors, vaccines, synthetic vectors, growth factors, neurotrophic factors, hormones, cytokines, enzymes and agents for targeted lesioning of specific sites.

17. The method according to claim 1, wherein said active agent is selected from the group consisting of nucleic acids, nucleic acid analogs, proteins, including antibodies, small molecule chemical compositions, agents that exhibit toxicity and unwanted effects when administered systemically, EGFR inhibitors, Tarceva, Iressa, topoisomerase inhibitors, irinotecan (CPT-11), etoposide, topotecan, edotecarin, rubitican, valrubicin, fostriecin, GL331, XR5000, SGN15, anthrcyclines, doxorubicin, alkylating agents, temaxolamide, carboplatin, cisplatin, dacarbazine (DTIC), mTOR inhibitors, Rapamycin, CCI-779, RAD 001, Farnasyl transferase inhibitors, R11577, lonafarnib; growth factor inhibitors, tyrosine kinase inhibitors, AEE788, SU5416, erlotinab, ZD1839, Enzastaurin, lapatinib, AP23573, sorafenib, STI571 (Gleevac), PTK787, vatalanib, semaxanib, PKI166, quercetin, BIBX1382, Mubritinib, Erbstatin, RG13022, RG13291, AG1295, leflunomide, Gefitinib, HDAC inhibitors, depsipetide, integrin inhibitors, celengitide, COX-2 inhibitors, everolimus, vioxx, celebrex, telomerase inhibitors, grn 163, TGFb inhibitors, MDMA inhibitors, AMPA inhibitors, GABA, GABA agonists, inhibitors of axonal sprouting, and combinations thereof, including combinations of mTOR inhibitor and tyrosine kinase inhibitor, which combinations may be in a single carrier.

18. A pharmaceutical composition, comprising:
a high molecular weight neurotherapeutic;
wherein said pharmaceutical composition is deliverable by CED to the CNS of a patient having a CNS disorder; and
wherein said high molecular weight neurotherapeutic is present in an amount sufficient to provide a therapeutically effective dose when said pharmaceutical composition is delivered by CED to the CNS of said patient.

19. The pharmaceutical composition according to claim 18, wherein said pharmaceutical composition is a component of a kit.

20. The pharmaceutical composition according to claim 19, wherein the kit comprises a delivery device.

21. The pharmaceutical composition according to claim 20, wherein the delivery device comprises a step-design reflux-free cannula.
